(19) United States
(12) Patent Application Publication
(10) Pub. No.: US 2022/0185852 A1
(43) Pub. Date: Jun. 16, 2022

(54) METHODS AND COMPOSITIONS FOR IMPROVED PRODUCTION OF AN ANTIGEN FOR USE IN AN S. AUREUS VACCINE

(71) Applicant: Bayer Animal Health GmbH, Leverkusen (DE)

(72) Inventors: Thomas ILG, Monheim (DE); Romina SCHAUER, Lenexa, KS (US)

(21) Appl. No.: 17/547,429

(22) Filed: Dec. 10, 2021

Related U.S. Application Data
(60) Provisional application No. 63/124,246, filed on Dec. 11, 2020.

(51) Int. Cl. C07K 14/31; A61P 31/04; C12N 15/63

(52) U.S. Cl. CPC C07K 14/31; C07K 2319/33; C12N 15/63; A61P 31/04

(57) ABSTRACT

The present invention relates to an improved antigen derived from Staphylococcus aureus and uses thereof against Staphylococcus infections, such as bovine intramammary infections. In particular, a modified SACOL1867 polypeptide is provided comprising a mutation at one or more amino acid positions, wherein at least one of the mutations disrupts the catalytic binding site and/or decreases proteolytic activity of the modified SACOL1867 polypeptide compared to a reference SACOL1867 polypeptide. Fusion polypeptides comprising a modified SACOL1867 polypeptide of the invention are also provided, as well as vaccine compositions comprising the modified SACOL1867 polypeptides and/or fusion polypeptides thereof.

### CROSS-REFERENCE TO RELATED APPLICATIONS

This application claims the benefit of U.S. Provisional Patent Application No. 63/124,246, filed 11 Dec. 2020, the contents of which are hereby incorporated by reference in its entity.

### REFERENCE TO SEQUENCE LISTING SUBMITTED AS A COMPLIANT ASCII TEXT FILE (.TXT)

Pursuant to the EFS-Web legal framework and 37 C.F.R. § 1.821-825 (see M.P.E.P. § 2442.03(a)), a Sequence Listing in the form of an ASCII-compliant text file (entitled “Sequence_listing_2920951-145001_ST25.txt” created on 6 Dec. 2021, and 180,808 bytes in size) is submitted concurrently with the instant application, and the entire contents of the Sequence Listing are incorporated herein by reference.

### BACKGROUND

### 1. Field

The present invention relates to an improved antigen derived from Staphylococcus aureus and uses thereof against Staphylococcus infections, such as bovine intramammary infections.

### 2. Description of Related Art

Bovine mastitis is one of the most common diseases in dairy cattle. Mastitis occurs when the udder becomes inflamed. Inflammation may be caused by many types of injury including infectious agents and their toxins, physical trauma or chemical irritants. Many microorganisms or bacteria have been identified as causing mastitis, but it is believed that serious cases of mastitis are in most instances caused by Staphylococcus aureus, Streptococcus agalactiae, Streptococcus dysgalactiae, Streptococcus uberis, and E. coli. Staphylococcus aureus in particular is the most often responsible for the development of mastitis.

The most common mastitis pathogens are found either in the udder (contagious pathogens) or the cow's surroundings (environmental pathogens). Contagious pathogens, such as Streptococcus agalactiae and Staphylococcus aureus, primarily colonize host tissue sites such as mammary glands, teat canals, and teat skin lesions, and are generally spread from infected udders to healthy udders during the milking process. This can include, but is not limited to, spread through contaminated teatcup liners, milker's hands, paper or cloth towels used to wash or dry more than one cow, and possibly flies. Environmental pathogens, such as streptococci, enterococci, and coliform organisms, are commonly present within the cow's surroundings from sources such as cow feces, soil, plant material, bedding, or water; and cause infection by casual opportunistic contact with an animal. In all cases of mastitis, whatever the causal microorganism, the route of transmission of the pathogen into the udder is through the teat orifice and teat canal.

Mastitis causes compositional changes in milk, including an increase in somatic cell count (SCC). Milk from normal (uninfected) cows generally contain below 200,000 somatic cells/ml. An elevation in SCC, above 300,000 somatic cells/ml is abnormal and is an indication of inflammation of the udder. The types of somatic cells present in the milk change to mostly white blood cells, which add many proteolytic and lipolytic enzymes to milk. In addition, more blood serum leaks into the milk than usual. Dairy product quality defects resulting from mastitis are due to enzymatic breakdown of milk protein and fat. Casein (the major milk protein of high nutritional quality) declines, and lower quality whey proteins increase, and this adversely impacts dairy product quality, such as cheese yield, flavor and quality. Protein breakdown in the milk can occur in milk from cows with clinical or subclinical mastitis due to the presence of proteolytic enzymes. Plasmin increases proteolytic activity more than 2-fold during mastitis. Plasmin and enzymes derived from somatic cells can cause damage to casein in the udder before milk removal. Deterioration of the milk protein may also continue during processing and storage of milk from infected cows. Other compositional changes in the milk include a decrease in potassium and calcium levels.

Mastitis costs the U.S. dairy industry about 1.7-2 billion dollars annually or 11% of the total U.S. milk production. The cost includes reduced milk production, discarded milk, replacement cows, medication, labor, and veterinary services. Currently, acute mastitis is treated with antibiotics, anti-inflammatories and oxytocin. The treatments, however, are often time-consuming (sometimes requiring several successive intramammary applications), expensive, and not fully efficacious. As such, there is a need for a prevention and control of Staphylococcal intramammary infections, especially intramammary infections caused by Staphylococcus aureus.

A lack of vaccine efficacy and protective ability has been noted for commercially available S. aureus vaccines. This has been postulated to be because the components of S. aureus these vaccines are directed to are not necessarily the components of S. aureus that are expressed during active intramammary infection.

U.S. Pat. No. 10,029,004 describes screening of S. aureus genes expressed during bovine intramammary infection to identify putative targets for vaccine development. In particular, 43 genes, such as SACOL1867, were identified to be significantly expressed during intramammary S. aureus infection in cattle.

However, attempts to recombinantly express SACOL1867 antigens for vaccine development have resulted in low yields and have been of low quality.

A solution to this technical problem is provided by the compositions and methods of the present invention.

### BRIEF SUMMARY

The present application provides a modified SACOL1867 polypeptide comprising a mutation at one or more amino acid positions, wherein at least one of the mutations disrupts the catalytic binding site and/or decreases proteolytic activity of the modified SACOL1867 polypeptide compared to a reference SACOL1867 polypeptide. In some embodiments, the modified SACOL1867 polypeptide comprises at least one mutation at a position corresponding to an amino acid residue within the catalytic triad of the SACOL1867 polypeptide sequence. In some embodiments, the modified SACOL1867 polypeptide comprises at least one mutation at a position corresponding to amino acid residue 75, 113, and/or 193 of SEQ ID NO:2, preferably at a position corresponding to amino acid residue 193 of SEQ ID NO:2.

In some embodiments, the at least one mutation is an amino acid substitution to any other amino acid not naturally occurring at His 75, Asp 113 and/or Ser 193 in the amino acid sequence of the reference (e.g., wild-type) SACOL1867 polypeptide. In some embodiments, the at least one mutation is a mutation of Ser 193 to an amino acid selected from: alanine; glycine; proline; isoleucine; leucine; and valine. In some embodiments, the at least one mutation is a mutation of Ser 193 to an amino acid selected from: cysteine; glycine; isoleucine; leucine; methionine; phenylalanine; proline; tryptophan; and valine. In some embodiments, the modified SACOL1867 polypeptide comprises a mutation at a position corresponding to amino acid residue 193 of SEQ ID NO:2 in which serine is replaced with alanine. In some embodiments, the modified SACOL1867 polypeptide comprises the amino acid sequence of SEQ ID NO:5 (full length SACOL1867), in which serine is replaced with alanine at position 193) or SEQ ID NO:6 (aa 41-239 of SEQ ID NO:5).

The present application also provides a fusion polypeptide of formula I:

X-A-linker-B—Z (formula I),

in which at least one of A or B is a modified SACOL1867 polypeptide of the invention and the other of A or B can be any polypeptide; the linker is an amino acid sequence of at least one amino acid or is absent; X is an amino acid sequence of at least one amino acid or is absent; and Z is an amino acid sequence of at least one amino acid or is absent.

In some embodiments, A is selected from a polypeptide comprising a SACOL0029 polypeptide as set forth in any one of SEQ ID NOS:19-30, a SACOL0264 polypeptide as set forth in SEQ ID NO:31, a SACOL0442 polypeptide as set forth in any one of SEQ ID NOS: 32-43, a SACOL0718 polypeptide as set forth in SEQ ID NO:44, a SACOL0720 polypeptide as set forth in any one of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of SEQ ID NOS: 2, 3, and 5-18, a SACOL1912 polypeptide as set forth in SEQ ID NO:61, a SACOL1944 polypeptide as set forth in SEQ ID NO:62, a SACOL2144 polypeptide as set forth in SEQ ID NO:63, a SACOL2365 polypeptide as set forth in SEQ ID NO:64, a SACOL2385 polypeptide as set forth in SEQ ID NO:65, or a SACOL2599 polypeptide as set forth in SEQ ID NO:66 (including fragments and/or variants of any of the above); and B is a modified SACOL1867 polypeptide of the invention. In a preferred embodiment, A is a SACOL0029 polypeptide as set forth in any one of SEQ ID NOS: 19-30, and B is a modified SACOL1867 polypeptide of the invention. In a more preferred embodiment, A is a SACOL0029 polypeptide as set forth in SEQ ID NO:19 and B is a modified SACOL1867 polypeptide as set forth in SEQ ID NO:6.

In some embodiments, there is no linker between polypeptide A and polypeptide B within the fusion polypeptide. In some embodiments, the linker is present between polypeptide A and polypeptide B. In some embodiments, the linker comprises or consists of (GGGGS)n (SEQ ID NO:76), (ERKYK)n (SEQ ID NO:70); or (EAAAK)n (SEQ ID NO:72), in which n is an integer from 1 to 5. In a preferred embodiment, the linker is an amino acid sequence selected from GGGGSGGGGSGGGGS (SEQ ID NO:69), ERKYK (SEQ ID NO:70), and EAAAKEAAAK (SEQ ID NO:71).

In some embodiments, the fusion polypeptide comprises or consists of the amino acid sequence of SEQ ID NO:67 (SACOL0029-SACOL1867mut).

The present application also provides a nucleic acid comprising a polynucleotide sequence encoding a modified SACOL1867 polypeptide of the invention or a fusion polypeptide comprising a modified SACOL1867 polypeptide of the invention. Also provided are vectors and/or host cells comprising the nucleic acid of the invention. In some embodiments, the vectors are expression vectors. In some embodiments, the host cell is a bacterial cell, such as an E. coli cell.

Methods for recombinantly expressing a modified SACOL1867 polypeptide and/or a fusion polypeptide comprising a modified SACOL1867 polypeptide are also provided comprising culturing a host cell comprising a nucleic acid and/or vector of the invention under conditions conducive for expression of the modified SACOL1867 polypeptide and/or fusion polypeptide comprising a modified SACOL1867 polypeptide.

Modified SACOL1867 polypeptides of the invention, including fusions thereof, produced by recombinant or other means can be used, for example, for production of vaccine compositions. Recombinant production of modified SACOL1867 polypeptides of the invention, including fusions thereof, preferably occurs in bacterial cells, more preferably in E. coli cells.

The present application also provides compositions comprising: (i) a modified SACOL1867 polypeptide, a fusion polypeptide comprising a modified SACOL1867 polypeptide, a nucleic acid, a vector, or a host cell of the invention; and (ii) a pharmaceutically acceptable excipient and/or an adjuvant.

Kits are also provided comprising: (i) a modified SACOL1867 polypeptide, a fusion polypeptide comprising a modified SACOL1867 polypeptide, a nucleic acid, a vector, a host cell, or a composition of the invention; and (ii) instructions for using the kit.

Also provided are methods for preventing, treating, and/or controlling a Staphylococcal infection in a subject comprising administering an effective amount of a modified SACOL1867 polypeptide, a fusion polypeptide comprising a modified SACOL1867 polypeptide, a nucleic acid, a vector, a host cell, or a composition of the invention. In some embodiments, the Staphylococcal infection is a S. aureus infection, such as an intramammary S. aureus infection. In some embodiments, the subject is a mammal, preferably a cow.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 shows a multiple sequence alignment of the full length wild-type SACOL1867 polynucleotide sequence (SEQ ID NO:1) with orthologous polynucleotide sequences (SEQ ID NOs: 84-94). “*” indicates residues that are identical in all sequences of the alignment.

FIG. 2 shows a multiple sequence alignment of the full length wild-type SACOL1867 polypeptide sequence (SEQ ID NO:2) with orthologous polypeptide sequences and consensus sequence derived therefrom (SEQ ID NOs: 7-18). “*” indicates residues that are identical in all sequences of the alignment. “:” indicates residues that are conserved in all sequences of the alignment. “.” indicates residues that are semi-conserved in all sequences of the alignment. Selected epitopes are rendered in boldface type and the end of the signal peptide and/or transmembrane domain is marked with a vertical line (separating signal peptide and/or transmembrane domain from mature and/or secreted form).

FIGS. 3A & 3B show protein expression of wild-type SACOL0029-1867 (FIG. 3A) and mutant SACOL0029-1867 (FIG. 3B) in E. coli. In particular, E. coli W3110 cells were transfected with a vector encoding either wild-type SACOL0029-1867 (SEQ ID NO:80) or mutant SACOL0029-1867 (SEQ ID NO:67). Expression of the wild-type or mutant SACOL0029-1867 antigen was induced by IPTG for either 4 hrs, 16 hrs, or 20 hrs. Insoluble (inclusion body) and soluble E. coli fractions were obtained following induction and analyzed by SDS-polyacrylamide gel. Non-induced (ni) samples were included as negative controls. Molecular weight markers were included in lanes labelled “M”. (FIG. 3A): Soluble wild-type SACOL0029-1867 is degraded to a form several thousand Da smaller than the expected product (top arrow=expected size; bottom arrow=observed size). This smaller, degraded SACOL0029-1867 product is incapable of binding to a hexa-His binding resin. In the insoluble fraction, the recombinant protein form with the expected size predominates, probably due to protection from protease action by inclusion body formation. (FIG. 3B): The size of the mutant SACOL0029-1867 antigen is the same in both insoluble and soluble fractions, and the soluble antigen binds to Ni2+-NTA or Ni2+-IDA resins. As in FIG. 3A, the top arrow indicates the expected size of the SACOL0029-1867 antigen and the bottom arrow indicates the size of the degraded fragment that was obtained from wild-type SACOL0029-1867 samples.

FIGS. 4A & 4B show results obtained from comparative indirect ELISA analyses performed using saturating amounts (5 μg/ml) of antigen (mutant SACOL0029-1867 or wild-type SACOL0029-1867) coated on polystyrene ELISA plates. FIG. 4A provides titration curves obtained using a specific anti-SACOL0029-1867 WT antiserum raised in rabbits. FIG. 4B provides titration curves obtained using serum obtained from a cow immunized against the wild-type SACOL0029-1867 antigen. Solid circles indicate results from mutant SACOL0029-1867 (SEQ ID NO:67); open squares indicate results from wild-type SACOL0029-1867 (SEQ ID NO:80).

FIGS. 5A-5C show results obtained from comparative indirect ELISA analyses performed using sub-saturating amounts (500 ng/ml) of antigen (mutant SACOL0029-1867 or wild-type SACOL0029-1867) coated on polystyrene ELISA plates. FIG. 5A provides titration curves obtained using a specific anti-SACOL0029-1867 WT antiserum raised in rabbits. FIG. 5B provides titration curves obtained using serum obtained from a cow immunized against the wild-type SACOL0029-1867 antigen. FIG. 5C provides titration curves obtained from a mouse (AD485) immunized against wild-type SACOL0029-1867 antigen. Solid circles indicate results from mutant SACOL0029-1867 (SEQ ID NO:67); open squares indicate results from wild-type SACOL0029-1867 (SEQ ID NO:80) (batch 1); closed triangle indicate results from wild-type SACOL0029-1867 (SEQ ID NO:80) (batch 2).

FIG. 6 shows serum and ELISA results from two groups of cows: one group was vaccinated with a vaccine formulation containing wild-type SACOL0029-1867 (cows #518, 603, 185, 203, 602, 9007), and the other group was vaccinated with a vaccine formulation containing mutant SACOL0029-1867* (cows #637, 228, 636). The adjuvant and vaccination schedule were the same for both groups of cows. The only difference was the utilization of wild-type SACOL0029-1867 (SEQ ID NO:80) vs. mutant SACOL0029-1867 (SEQ ID NO:67).

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

In this specification and the appended claims, the singular forms “a,” “an,” and “the” include plural reference unless the context clearly dictates otherwise.

The “SACOL” nomenclature used throughout the disclosure is based on the gene nomenclature from the Staphylococcus aureus subsp. aureus COL (SACOL) complete genome set forth in NCBI Reference Sequence NC_002951.2, in which the digits following “SACOL” refer to a particular SACOL gene.

Unless defined otherwise, all technical and scientific terms used herein have the same meaning as commonly understood to one of ordinary skill in the art to which this disclosure belongs.

The present application relates to modified SACOL1867 (also referred to as “SA1867” or “SACOL_RD09575”; see NCBI Accession No. NC_002951.2) polypeptides (see, e.g., NCBI Accession No. WP_001038867.1) mutated to improve recombinant production and/or stability and their use in vaccine compositions. In particular, the present invention provides modified SACOL1867 polypeptides mutated at one or more positions to disrupt the catalytic binding site and/or to decrease proteolytic activity of SACOL1867. For example, the modified SACOL1867 polypeptides of the invention may be mutated at one or more positions corresponding to the catalytic triad (e.g., at one or more positions corresponding to residue(s) 75, 113, and/or 193 of SEQ ID NO:2).

In a preferred embodiment, the modified SACOL1867 polypeptide or fragment thereof comprises a mutation at a position corresponding to amino acid residue 193 of SEQ ID NO:2. In some embodiments, the modified SACOL1867 polypeptide or fragment thereof is mutated at a position corresponding to serine 193 of SEQ ID NO:2 to any other amino acid. In some embodiments, the modified SACOL1867 polypeptide or fragment thereof is mutated at a position corresponding to amino acid residue 193 of SEQ ID NO:2 from serine to alanine, glycine, proline, isoleucine, leucine, or valine. In some embodiments, the modified SACOL1867 polypeptide or fragment thereof is mutated at a position corresponding to amino acid residue 193 of SEQ ID NO:2 from serine to cysteine, glycine, isoleucine, leucine, methionine, phenylalanine, proline, tryptophan, or valine. In some embodiments, the modified SACOL1867 polypeptide or fragment thereof is mutated at a position corresponding to amino acid residue 193 of SEQ ID NO:2 from serine to an alanine (S193A). In some embodiments, the modified SACOL1867 polypeptide is the polypeptide of SEQ ID NO:5 (identical to SEQ ID NO:2 but bearing a S193A mutation) or SEQ ID NO:6 (identical to SEQ ID NO:5, but with the transmembrane domain (amino acids 1-40 of SEQ ID NO:2) deleted), a fragment of SEQ ID NO: 5 or 6, or a variant of SEQ ID NO: 5 or 6.

In other embodiments, the modified SACOL1867 polypeptide or fragment thereof is mutated at one or more positions outside of the catalytic triad (e.g., at a position other than a position corresponding to amino acid residue(s) 75, 113, and/or 193 of SEQ ID NO:2) such that the catalytic triad is disrupted and/or protease activity is reduced or eliminated.

Variants

Useful SACOL1867 antigens can elicit generation of an antibody (e.g., when administered to a mammal) that recognizes SEQ ID NO:2 or fragments thereof and/or which may comprise an amino acid sequence: (a) having 50% or more identity (e.g., 60%, 65%, 70%, 75%, 80%, 85%, 90%, 91%, 92%, 93%, 94%, 95%, 96%, 97%, 98%, 99%, 99.5% or more) to any of SEQ ID NOs: 2, 3, 5, and 6; and/or (b) comprising a fragment of at least ‘n’ consecutive amino acids of any of SEQ ID NOs: 2, 3, 5, and 6, wherein ‘n’ is an integer value of 7 or more (e.g., 8, 10, 12, 14, 16, 18, 20, 25, 30, 35 35, 40, 50, 60, 70, 80, 90, 100, 150, 200 or more)—which may be referred to herein as “variants”. These variant SACOL1867 polypeptides include variants of SEQ ID NOs: 5 or 6. In a preferred embodiment, the SACOL1867 polypeptide variant is the variant having the amino acid sequence of SEQ ID NO: 5 or 6 (SACOL1876mut).

In some embodiments, the modified SACOL1867 polypeptide of SEQ ID NO: 5 or 6 comprises one or more further mutations.

Preferred fragments of (b) comprise at least 4 consecutive amino acids from any of SEQ ID NOs: 2, 3, 5, and 6, preferably between 4 to 20 consecutive amino acids from any of SEQ ID NOs: 2, 3, 5, and 6. Other preferred fragments lack one or more amino acids (e.g., 1, 2, 3, 4, 5, 6, 7, 8, 9, 10, 15, 20, 25 or more) from the C-terminus and/or one or more amino acids (e.g., 1, 2, 3, 4, 5, 6, 7, 8, 9, 10, 15, 20, 25 or more) from the N-terminus of any of SEQ ID NOs: 2, 3, 5, and 6 while retaining at least one epitope of SEQ ID NO: 2, 3, 5, and 6.

Fragments of (b) also include immunogenic fragments. An immunogenic fragment of a protein/polypeptide preferably comprises one or more epitopes of said protein/polypeptide. An epitope of a protein/polypeptide is defined as a fragment of said protein/polypeptide of at least about 4 or 5 amino acids in length, capable of eliciting a specific antibody and/or an immune cell (e.g., a T cell or B cell) bearing a receptor capable of specifically binding said epitope. Two different kinds of epitopes exist: linear epitopes and conformational epitopes. A linear epitope comprises a stretch of consecutive amino acids. A conformational epitope is typically formed by several stretches of consecutive amino acids that are folded in position and together form an epitope in a properly folded protein. An immunogenic fragment as used herein refers to either one, or both, of said types of epitopes. In an embodiment where immunogenic fragments are used alone (i.e., not fused in a larger polypeptide construct (e.g., fusion with other antigenic fragment)), the immunogenic fragment of a protein/polypeptide comprises at least 16 amino acid residues. In some embodiments, the immunogenic fragment comprises at least 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 110, 120, 130, 140, 150, or 160 consecutive amino acids of the protein/polypeptide. In a specific embodiment, the fragment has at least 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 50 or more consecutive amino acids of the protein/polypeptide. In an embodiment where the at least one immunogenic fragment forms part of a larger polypeptide construct (e.g., fusion with other antigenic polypeptide, fragment or variant thereof), the immunogenic fragment comprises at least 13 consecutive amino acid residues of the polypeptide.

Preferred fragments are secreted or extracellular fragments, i.e., SACOL1867 in which the transmembrane domain has been removed. For example, amino acids 1-40 of SACOL1867 (SEQ ID NO:2) have been predicted to be a transmembrane domain such that the extracellular domain would include amino acid positions 41-239. However, other predictive software has predicted SACOL1867 to be an extracellular protein. Since the above-mentioned transmembrane and/or signal peptide domains are putative, the present invention encompasses cases where the antigens presented herein (e.g., SACOL1867) do or do not have a signal peptide and/or transmembrane domain and encompasses the corresponding extracellular fragments (e.g., SEQ ID NOs: 3 and 6). In some embodiments, the above-mentioned polypeptide is secreted or expressed at the surface of the bacteria (e.g., Staphylococcus aureus).

In some embodiments, the modified SACOL1867 polypeptide can be any orthologous sequence described herein (SEQ ID NOs: 7-18) comprising a mutation that reduces or prevents protease activity. In some embodiments, the modified SACOL1867 polypeptide can be any orthologous sequence described herein (SEQ ID NOs: 7-18) comprising a mutation at a position corresponding to amino acid position 193 of SEQ ID NO:2. Table 1 describes the SACOL1867 polypeptide and polynucleotide sequences provided herein.

TABLE 1

| SEQ | | | |
|---|---|---|---|
| ID NO: | Gene Name | S. Aureus strain | Description |
| 1 | SACOL1867 | COL | nt sequence encoding full length SpIC protein |
| | | | (Genbank Gene ID: 3236101) |
| 2 | SACOL1867 | COL | full length SpIC protein (Genbank Accession |
| | | | No: AAW36882; 239 aa) |
| 3 | SACOL1867 | COL | truncated SpIC protein (Genbank Accession |
| | | | No: AAW36882; 41-239 aa) |
| 5 | SACOL1867mut | COL | full length S193A mutant SACOL1867 protein |
| | | | (239 aa) |
| 6 | SACOL1867mut | COL | truncated S193A mutant SACOL1867 protein |
| | | | (41-239 aa) |
| 7 | MW1753 | MW2 | serine protease SpIC (Genbank Accession |
| | | | No. BAB95618.1) |
| 8 | SAS1734 | MSSA476 | serine protease (Genbank Accession No. |
| | | | CAG43538.1) |
| 9 | SAOUHSC_01939 | NCTC8325 | serine protease SpIC (Genbank Accession |
| | | | No. YP_500440.1) |
| 10 | NWMN_1704 | Newman | serine protease SpIC (Genbank Accession |
| | | | No. BAF67976.1) |
| 11 | SAUSA300_1756 | USA300_FPR3757 | serine protease SpIC (Genbank Accession |
| | | | No. ABD21391.1) |
| 12 | SaurJH1_1897 | JH1 | peptidase S1 and S6 chymotrypsin/Hap |
| | | | (Genbank Accession No. ABR52735.1) |
| 13 | SAHV_1796 | Mu3 | serine protease (Genbank Accession No. |
| | | | BAF78679.1) |
| 14 | SaurJH9_1862 | JH9 | peptidase S1 and S6, chymotrypsin/Hap |
| | | | (Genbank Accession No. ABQ49649.1) |
| 15 | SAV1811 | Mu50 | serine protease (Genbank Accession No. |
| | | | BAB57973.1) |
| 16 | SA1629 | N315 | serine protease SpIC (Genbank Accession |
| | | | No. BAB42897.1) |
| 17 | SAB1671c | RF122 | serine protease (Genbank Accession No. |
| | | | CAI81360.1) |
| 18 | N/A | N/A | consensus sequence from alignment of SEQ |
| | | | ID NOs: 2 & 7-17 |
| 84 | MW1753 | MW2 | nt sequence encoding serine protease SpIC |
| | | | (Genbank Accession No. NC_003923.1) |
| 85 | SAS1734 | MSSA476 | nt sequence encoding serine protease |
| | | | (Genbank Accession No. NC_002953.3) |
| 86 | SAOUHSC_01939 | NCTC8325 | nt sequence encoding serine protease SpIC |
| | | | (Genbank Accession No. NC_007795.1) |
| 87 | NWMN_1704 | Newman | nt sequence encoding serine protease SpIC |
| | | | (Genbank Accession No. NC_009641.1) |
| 88 | SAUSA300_1756 | USA300_FPR3757 | nt sequence encoding serine protease SpIC |
| | | | (Genbank Accession No. NC_007793.1) |
| 89 | SaurJH1_1897 | JH1 | nt sequence encoding peptidase S1 and S6 |
| | | | chymotrypsin/Hap (Genbank Accession No. |
| | | | NC_009632.1) |
| 90 | SAHV_1796 | Mu3 | nt sequence encoding serine protease |
| | | | (Genbank Accession No. NC_009782.1) |
| 91 | SaurJH9_1862 | JH9 | nt sequence encoding peptidase S1 and S6, |
| | | | chymotrypsin/Hap (Genbank Accession No. |
| | | | NC_009487.1) |
| 92 | SAV1811 | Mu50 | nt sequence encoding serine protease |
| | | | (Genbank Accession No. NC_002758.2) |
| 93 | SA1629 | N315 | nt sequence encoding serine protease SpIC |
| | | | (Genbank Accession No. NC_002745.2) |
| 94 | SAB1671c | RF122 | nt sequence encoding serine protease |
| | | | (Genbank Accession No. NC_007622.1) |

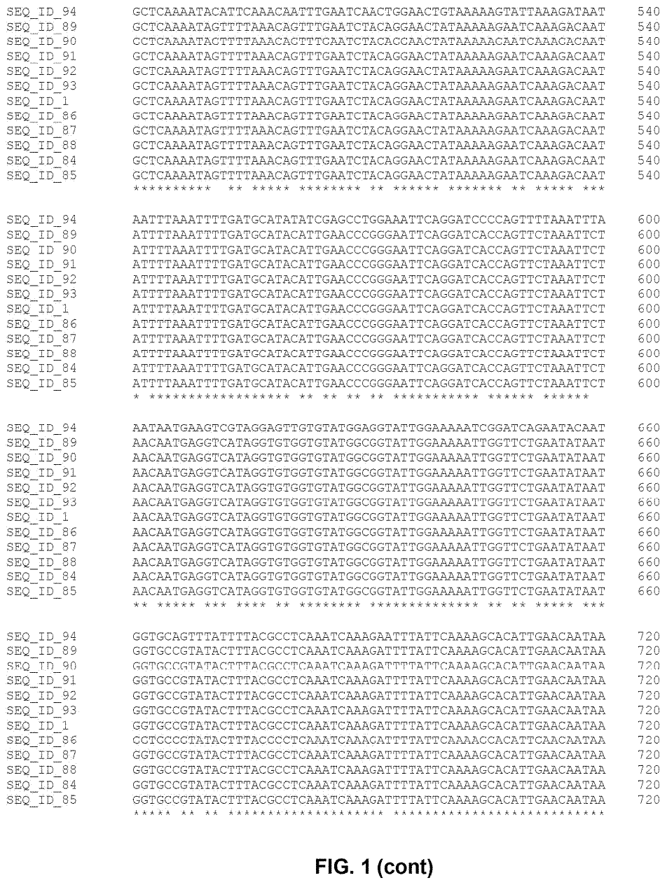

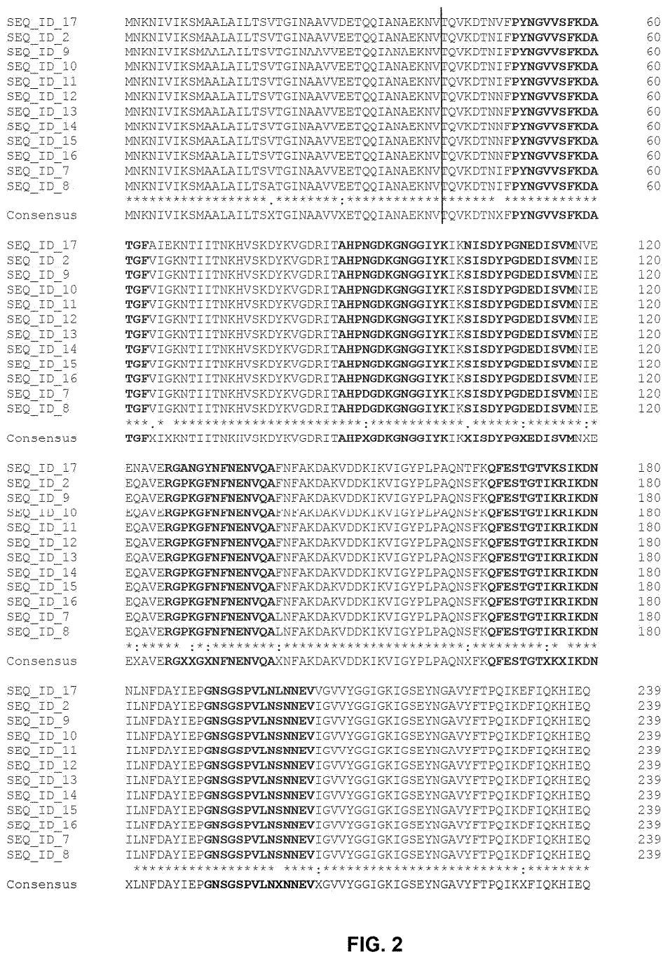

Each “X” in the consensus sequence (FIG. 2) is defined as being any amino acid, or absent when this position is absent in one or more of the orthologues presented in the alignment. In some embodiments, each “X” in the consensus sequences is defined as being any amino acid that constitutes a conserved or semi-conserved substitution of any of the amino acid in the corresponding position in the orthologues presented in the alignment, or absent when this position is absent in one or more of the orthologues presented in the alignment. In FIG. 2, conservative substitutions are denoted by the symbol “:” and semi-conservative substitutions are denoted by the symbol “.”. In another embodiment, each “X” refers to any amino acid belonging to the same class as any of the amino acid residues in the corresponding position in the orthologues presented in the alignment, or absent when this position is absent in one or more of the orthologues presented in the alignment. In another embodiment, each “X” refers to any amino acid in the corresponding position of the orthologues presented in the alignment, or absent when this position is absent in one or more of the orthologues presented in the alignment.

Conservative amino acid mutations may be made to generate polypeptide variants of the invention. Conservative amino acid mutations may include addition, deletion, or substitution of an amino acid; a conservative amino acid substitution is defined herein as the substitution of an amino acid residue for another amino acid residue with similar chemical properties (e.g., size, charge, or polarity). Such a conservative amino acid substitution may be a substitution of a basic, neutral, hydrophobic, or acidic amino acid for another of the same group. By the term “basic amino acid,” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gin or Q). The term “hydrophobic amino acid” (also “non-polar amino acid”), is meant to include amino acids exhibiting a hydrophobicity index of greater than zero. Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pK value of less than 7 which are typically negatively charged at physiological pH. Acidic amino acids include glutamate (Glu or E), and aspartate (Asp or D).

A semi-conserved amino acid substitution replaces one residue with another one that has similar steric conformation, but does not share chemical properties. Examples of semi-conservative substitutions would include substituting cysteine for alanine or leucine; substituting serine for asparagine; substituting valine for threonine; or substituting proline for alanine.

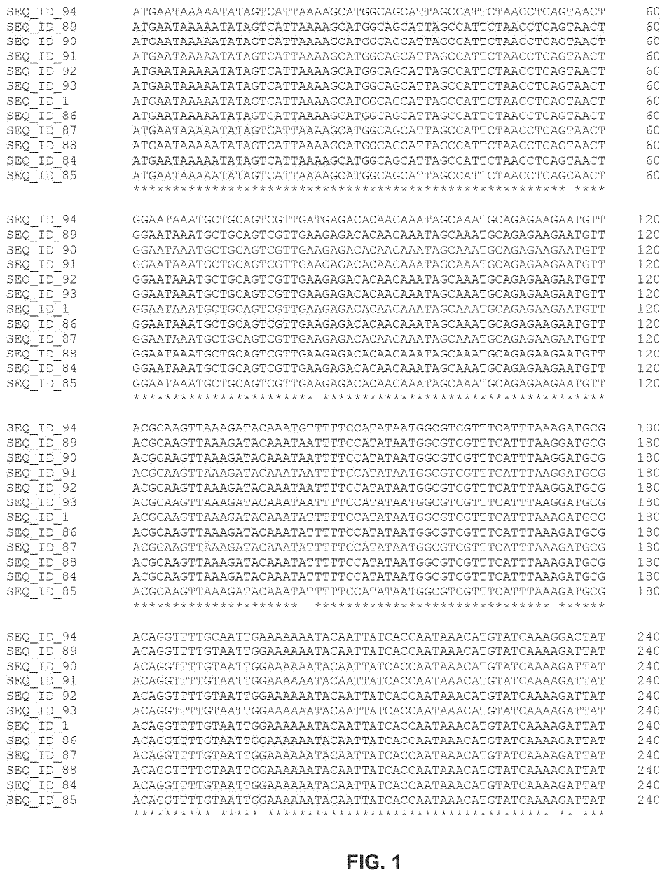

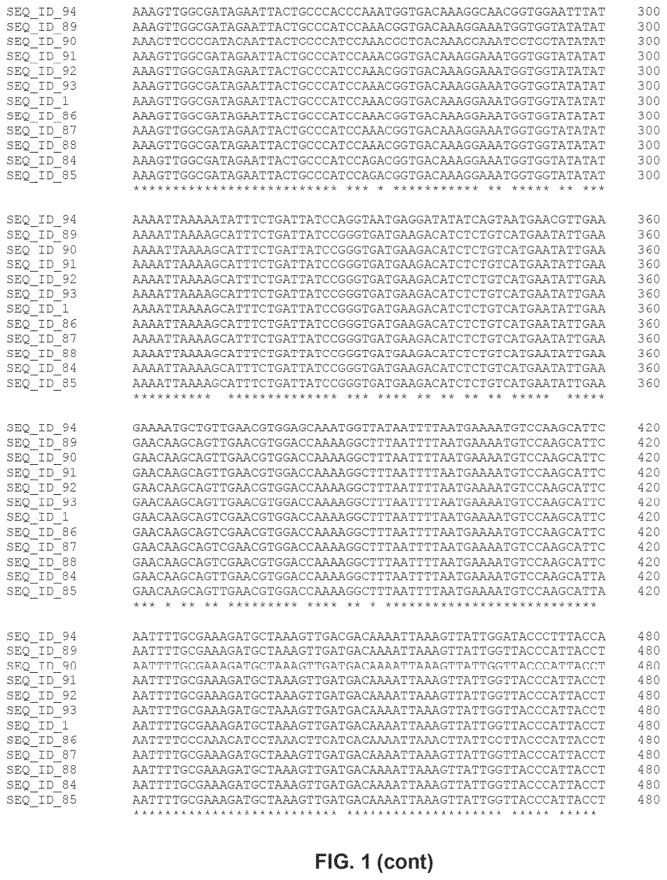

The similarity and identity between amino acid or nucleotide sequences can be determined by comparing each position in the aligned sequences. Optimal alignment of sequences for comparisons of similarity and/or identity may be conducted using a variety of algorithms, for example using a multiple sequence alignment program/software well known in the art such as ClustalW™, SAGA™, UGENE™ or T-coffee™. Examples of multiple sequence alignments are described herein and depicted in FIGS. 1 & 2.

Other examples of variants include antigens described herein (e.g., modified SACOL1867 polypeptides, or fusions, variants, and/or fragments thereof) comprising at either of their N- or C-termini or inserted within their amino acid sequence, an oligopeptide useful for purification (e.g., affinity purification) or useful as a spacer or linker. Examples of oligopeptides useful for affinity purification include, but are not limited to, polyhistidine tags (e.g., 6-10 histidine residues including or not RGS tags (e.g., HHHHHH, RGSHHHHHH, or RGSHHHHHGS; SEQ ID NOs: 81-83). The polyhistidine-tag may also be followed by an amino acid sequence suitable to facilitate a removal of the polyhistidine-tag using endopeptidases. The “X” and/or “Z” segments as recited in formulas (I)-(III) described below also may comprise such an oligopeptide useful for purification and/or a sequence suitable to facilitate removal of such an oligopeptide useful for purification.

Fusion Polypeptides

It has been shown that fusion of two or more antigens can synergistically improve immunological response. In some embodiments, the modified SACOL1867 polypeptide is fused with one or more other antigens. In some embodiments, the modified SACOL1867 polypeptide is fused with one or more other antigens useful for the prevention and or treatment of intramammary infections. In some embodiments, the modified SACOL1867 polypeptide is fused with one or more other antigens useful for the prevention and/or treatment of intramammary infections caused by Staphylococcus aureus. In some embodiments, the one or more other antigens are one or more other antigens derived from Staphylococcus aureus, in particular, one or more other SACOL antigens.

In some embodiments of the invention, a fusion polypeptide of formula I is provided:

X-A-linker-B—Z (formula I),

wherein A and/or B, preferably B, is a modified SACOL1867 polypeptide described herein, including fragments and/or variants thereof.

In some embodiments, A and/or B, preferably B, is the modified SACOL1867 polypeptide of SEQ ID NO:5 or a fragment and/or variant thereof. In a preferred embodiment, A and/or B, preferably B, is the modified SACOL1867 polypeptide of SEQ ID NO:5 in which the signal sequence and/or transmembrane domain has been removed, such as, for example, the modified SACOL1867 polypeptide of SEQ ID NO:6 (41-239 of SACOL1867mut).

In some embodiments, A is selected from (a) a polypeptide comprising a SACOL0029 polypeptide as set forth in any one of the sequences of SEQ ID NOS:19-30, a SACOL0264 polypeptide as set forth in SEQ ID NO:31, a SACOL0442 polypeptide as set forth in any one of the sequences of SEQ ID NOS: 32-43, a SACOL0718 polypeptide as set forth in SEQ ID NO:44, a SACOL0720 polypeptide as set forth in any one of the sequences of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of the sequences of SEQ ID NOS: 2, 3, and 5-18, a SACOL1912 polypeptide as set forth in SEQ ID NO:61, a SACOL1944 polypeptide as set forth in SEQ ID NO:62, a SACOL2144 polypeptide as set forth in SEQ ID NO:63, a SACOL2365 polypeptide as set forth in SEQ ID NO:64, a SACOL2385 polypeptide as set forth in SEQ ID NO:65, or a SACOL2599 polypeptide as set forth in SEQ ID NO:66, (b) a polypeptide encoded by a gene from a same operon as a gene encoding the polypeptide of (a); (c) a polypeptide comprising an immunogenic fragment of at least 13 consecutive amino acids of (a) or (b); (d) a polypeptide comprising an amino acid sequence at least 60% identical overall to the sequence of the polypeptide of any one of (a) to (c); or (e) a polypeptide comprising an immunogenic variant comprising at least 13 consecutive amino acids of any one of (a) to (c); and B is a modified SACOL1867 polypeptide as described herein, including fragments and/or variants thereof.

In another embodiment, A is a modified SACOL1867 polypeptide as described herein, including fragments and/or variants thereof; and B is selected from (a) a polypeptide comprising a SACOL0029 polypeptide as set forth in any one of the sequences of SEQ ID NOS: 19-30, a SACOL0264 polypeptide as set forth in SEQ ID NO:31, a SACOL0442 polypeptide as set forth in any one of the sequences of SEQ ID NOS: 32-43, a SACOL0718 polypeptide as set forth in SEQ ID NO:44, a SACOL0720 polypeptide as set forth in any one of the sequences of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of the sequences of SEQ ID NOS: 2, 3, and 5-18, a SACOL1912 polypeptide as set forth in SEQ ID NO:61, a SACOL1944 polypeptide as set forth in SEQ ID NO: 62, a SACOL2144 polypeptide as set forth in SEQ ID NO: 63, a SACOL2365 polypeptide as set forth in SEQ ID NO: 64, a SACOL2385 polypeptide as set forth in SEQ ID NO: 65, or a SACOL2599 polypeptide as set forth in SEQ ID NO: 66, (b) a polypeptide encoded by a gene from a same operon as a gene encoding the polypeptide of (a); (c) a polypeptide comprising an immunogenic fragment of at least 13 consecutive amino acids of (a) or (b); (d) a polypeptide comprising an amino acid sequence at least 60% identical overall to the sequence of the polypeptide of any one of (a) to (c); or (e) a polypeptide comprising an immunogenic variant comprising at least 13 consecutive amino acids of any one of (a) to (c).

In some embodiments, the above polypeptide A and/or B is the secreted or extracellular fragment of the polypeptide defined above. Transmembrane domains can be predicted using, for example, the software TMPRED™ (ExPASy) ch.embnet.org/software/TMPRED_form.html, psort.org/psortb/index.html, enzim.hu/hmmtop/html/submit.html and/or SignalP 4.1 (cbs.dtu.dk/services/SignalP). TMPRED™ and SignalP 4.1 predicted extracellular domains for: SACOL0720 (AA 310-508) and SACOL0442 (AA 36-203). Enzim predicted a transmembrane domain for SACOL1867 (1-40) such that the extracellular domain was AA 41-239 while psort.org/psortb/index.html predicted that SACOL1867 was an extracellular protein. Since the above-mentioned transmembrane and/or signal peptide domains are putative, the present invention encompasses cases where the antigens presented herein (e.g., SACOL1867) have or do not have a signal peptide and/or transmembrane domain and encompasses the corresponding extracellular fragments. In some embodiments, the above-mentioned polypeptide is a polypeptide normally secreted or expressed at the surface of the bacteria (e.g., Staphylococcus aureus).

In some embodiments, at least one modified SACOL1867 polypeptide of the invention, including fragments and/or variants thereof, is fused with a SACOL0029 polypeptide as set forth in any one of the sequences of SEQ ID NOS: 19-30. In some embodiments, the at least one modified SACOL1867 polypeptide of the invention is fused to the C-terminus of a SACOL0029 polypeptide as set forth in any one of the sequences of SEQ ID NOS: 19-30 (e.g., X-SACOL0029-linker-SACOL1867mut-Z). In another embodiment, the at least one modified SACOL1867 polypeptide of the invention is fused to the N-terminus of a SACOL0029 polypeptide as set forth in any one of the sequences of SEQ ID NOS: 19-30 (e.g., X-SACOL1867mut-linker-SACOL0029-Z).

In some embodiments, the modified SACOL1867 polypeptide of SEQ ID NO: 5 or 6, or a fragment and/or variant thereof, is fused to either the C-terminus or the N-terminus of a SACOL0029 polypeptide as set forth in any one of the sequences of SEQ ID NOS: 19-30. In a preferred embodiment, the modified SACOL1867 polypeptide of SEQ ID NO:5 in which the signal sequence and/or transmembrane domain has been removed, such as, for example, the modified SACOL1867 polypeptide of SEQ ID NO:6 (41-239 of SACOL1867mut) is fused to either the C-terminus or the N-terminus of a SACOL0029 polypeptide as set forth in any one of the sequences of SEQ ID NOS: 19-30, preferably SEQ ID NO:19.

In a preferred embodiment, a SACOL0029 polypeptide as set forth in any one of the sequences of SEQ ID NOS: 19-30, preferably SEQ ID NO:19, is fused to the modified SACOL1867 polypeptide of SEQ ID NO:6. A preferred SACOL0029-SACOL1867mut fusion polypeptide is provided in the amino acid sequence of SEQ ID NO:67.

Linkers

Insertion of linkers between fusion protein domains can increase bioactivity by augmenting distance between domains alleviating potential repulsive forces between different segments (e.g., antigenic fragments) of the fused polypeptide resulting in improved and/or restored protein folding. Different sequences of polypeptide linkers can be used and are known to have distinct properties, such as flexible, rigid, or cleavable linkers. The present invention encompasses the use of any such linkers including, but not limited to, any one of those listed in Chen et al., Adv Drug Deliv Rev. (2013); 65(10):1357-69, for example. Examples herein provide illustrations of specific linkers that have been used (i.e. GGGGSGGGGSGGGGS (SEQ ID NO:69), ERKYK (SEQ ID NO:70), and EAAAKEAAAK (SEQ ID NO:71)), i.e. flexible linker structures, rich in small hydrophilic amino acids, that maintain distance between the two connected domains and improve their folding.

Linkers may be included between contiguous antigens of the fusion polypeptide (e.g., one linker in a fusion comprising two antigens, two linkers in a fusion comprising three antigens, three linkers in a fusion comprising four antigens, etc.). In fusion polypeptides where large protein domains are used, the linker may be larger and may comprise a fragment crystallizable (Fc) region.

In some embodiments, the Fc region comprises a heavy chain constant domain 2 (CH2) domain, a heavy chain constant domain 3 (CH3) domain and a hinge region. In another embodiment, the Fc region is a constant domain of an immunoglobulin selected from the group consisting of IgG-1, IgG-2, IgG-3, IgG-3 and IgG-4. In another embodiment, the Fc region is a constant domain of an IgG-1 immunoglobulin.

In some embodiments, the linker is an amino acid sequence of at least one amino acid or is absent.

In some embodiments, the linker comprises at least three (e.g., at least 4, 5, 6, 7, 8, 9 or 10) amino acids selected from the group consisting of glycine, serine, alanine, aspartate, glutamate and lysine. In a specific embodiment, the linker is (EAAAK)n (SEQ ID NO:72); (GGGGS)n (SEQ ID NO:76); or (XPXPXP)n (SEQ ID NO:78) wherein “X” is any amino acid; wherein “n” is any integer from 1 to 5, more specifically 1, 2, 3, 4 or 5; EAAAKEAAAK (SEQ ID NO:71); EAAAKEAAAKEAAAK (SEQ ID NO:73); GGGGS (SEQ ID NO:76); GGGGSGGGGS (SEQ ID NO:77); GGGGSGGGGSGGGGS (SEQ ID NO:69); XPXPXP (SEQ ID NO:78), wherein “X” is any amino acid; XPXPXPXPXPXP (SEQ ID NO:79), wherein “X” is any amino acid; ERKYK (SEQ ID NO:70); ERKYKERKYK (SEQ ID NO:74); or ERKYKERKYKERKYK (SEQ ID NO:75). In a more specific embodiment, the linker is GGGGSGGGGSGGGGS (SEQ ID NO:69), ERKYK (SEQ ID NO:70), or EAAAKEAAAK (SEQ ID NO:71).

N- and C-Termini of Fusion Polypeptides

X and Z of formula (I) are each independently absent or an amino acid sequence of at least one amino acid. X and/or Z may be, for example, one or more of amino acids resulting from cloning strategy, amino acids used to facilitate purification of the fusion polypeptide (e.g., polyhistidine), and/or amino acids suitable to facilitate removal of a purification-tag using endopeptidases. In some embodiments, where the fusion polypeptide comprises three or more antigen polypeptides, any one of X and/or Z may also include the sequence of a further antigen (antigen C, antigen D, etc.) and, optionally, that of at least one further linker. In such embodiments where X and/or Z comprise one or more further antigen(s) and optionally linker(s), the fusion polypeptide could be more specifically illustrated as e.g., formula (II) or (III) as follows:
X′—C-linker1-A-linker2-B—Z′ (formula II) when the fusion comprises at least 3 antigens; or
X′—C-linker1-A-linker2-B-linker3-D-Z′ (formula III) when the fusion comprises at least 4 antigens.

In both formula (II) and formula (III), X′, Z′, linker1, linker2, and, optionally, linker3, are identical or different and are independently defined as are X, Z and linker in formula (I) defined herein.

In some embodiments, the fusion polypeptide comprises 2, 3, 4 or more antigen polypeptides (and further linkers). In some embodiments, and the fusion polypeptide is selected from, but not limited to, SACOL0029-SACOL1867mut; SACOL0029-SACOL0720-SACOL1867mut; SACOL0029-SACOL1867mut-SACOL0442; SACOL0442-SACOL0029-SACOL1867mut; SACOL0442-SACOL1867mut-SACOL0720; SACOL0720-SACOL0442-SACOL1867mut; or SACOL0029-SACOL1867mut-SACOL0720-SACOL0442, or any of the foregoing fusions wherein the antigen polypeptides are in any other order.

Polypeptide Combinations

The polypeptides of the present invention may be used as the sole immunogenic component of a composition (e.g., vaccine) of the present invention or in combination with one or more further fusion polypeptides(s), immunogenic polypeptide(s), fragment(s) or variant(s) thereof and/or live attenuated bacteria (e.g., S. aureus) (optionally expressing fusion polypeptide(s) and/or polypeptide(s), fragment(s) or variant(s) thereof).

The one or more further fusion polypeptide may be any immunogenic fusion polypeptide including a further fusion polypeptide as defined above. The one or more further fusion polypeptide may comprise any SACOL polypeptide described herein and any combination thereof. In some embodiments, the one or more further fusion polypeptide follow any one of formulas (I)-(III) outlined above, in which A and B are identical or different and are independently selected from any SACOL polypeptide described herein. For example, A and/or B may be identical or different and selected from (a) a polypeptide comprising a SACOL0029 polypeptide as set forth in any one of the sequences of SEQ ID NOS: 19-30, a SACOL0264 polypeptide as set forth in SEQ ID NO:31, a SACOL0442 polypeptide as set forth in any one of the sequences of SEQ ID NOS: 32-43, a SACOL0718 polypeptide as set forth in SEQ ID NO:44, a SACOL0720 polypeptide as set forth in any one of the sequences of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of the sequences of SEQ ID NOS: 2, 3, and 5-18, a SACOL1912 polypeptide as set forth in SEQ ID NO:61, a SACOL1944 polypeptide as set forth in SEQ ID NO:62, a SACOL2144 polypeptide as set forth in SEQ ID NO:63, a SACOL2365 polypeptide as set forth in SEQ ID NO:64, a SACOL2385 polypeptide as set forth in SEQ ID NO:65, or a SACOL2599 polypeptide as set forth in SEQ ID NO:66; (b) a polypeptide encoded by a gene from a same operon as a gene encoding the polypeptide of (a); (c) a polypeptide comprising an immunogenic fragment of at least 13 consecutive amino acids of (a) or (b); (d) a polypeptide comprising an amino acid sequence at least 60% identical overall to the sequence of the polypeptide of any one of (a) to (c); or (e) a polypeptide comprising an immunogenic variant comprising at least 13 consecutive amino acids of any one of (a) to (c).

The one or more immunogenic polypeptide(s), fragment(s) or variant(s) thereof for use in compositions of the present invention may be any polypeptide(s), fragment(s) or variant(s) that contribute to the immunogenicity of the compositions of the present invention as defined herein. Such polypeptide(s), fragment(s) or variant(s) includes, but are not limited to: (a) a SACOL0029 polypeptide as set forth in any one of the sequences of SEQ ID NOS: 19-30, a SACOL0264 polypeptide as set forth in SEQ ID NO:31, a SACOL0442 polypeptide as set forth in any one of the sequences of SEQ ID NOS: 32-43, a SACOL0718 polypeptide as set forth in SEQ ID NO:44, a SACOL0720 polypeptide as set forth in any one of the sequences of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of the sequences of SEQ ID NOS: 2, 3, and 5-18, a SACOL1912 polypeptide as set forth in SEQ ID NO:61, a SACOL1944 polypeptide as set forth in SEQ ID NO:62, a SACOL2144 polypeptide as set forth in SEQ ID NO:63, a SACOL2365 polypeptide as set forth in SEQ ID NO:64, a SACOL2385 polypeptide as set forth in SEQ ID NO:65, or a SACOL2599 polypeptide as set forth in SEQ ID NO:66; (b) a polypeptide encoded by a gene from a same operon as a gene encoding the polypeptide of (a); (c) a polypeptide comprising an immunogenic fragment of at least 13 consecutive amino acids of (a) or (b); (d) a polypeptide comprising an amino acid sequence at least 60% identical overall to the sequence of the polypeptide of any one of (a) to (c); or (e) a polypeptide comprising an immunogenic variant comprising at least 13 consecutive amino acids of any one of (a) to (c), as defined above.

The polypeptides of the invention described above can take various forms (e.g., native, fusions, glycosylated, non-glycosylated, lipidated, non-lipidated, phosphorylated, non-phosphorylated, myristoylated, non-myristoylated, monomeric, multimeric, particulate, denatured, etc.).

The polypeptides of the invention can be prepared by various means (e.g., recombinant expression, purification from cell culture, chemical synthesis, etc.). Recombinantly-expressed proteins are preferred, particularly for fusion polypeptides.

The polypeptides of the invention are preferably provided in purified or substantially purified form, i.e., substantially free from other polypeptides (e.g., free from naturally-occurring polypeptides), particularly from other staphylococcal or host cell polypeptides, and are generally at least about 50% pure (by weight), and usually at least about 90% pure, i.e., less than about 50%, and more preferably less than about 10% (e.g., 5%) of a composition is made up of other expressed polypeptides. Thus, the antigens in the compositions are separated from the whole organism from which the molecule is expressed.

The term “polypeptide” refers to amino acid polymers of any length. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention also provides a process for producing a polypeptide of the invention, comprising the step of culturing a host cell transformed with nucleic acid of the invention under conditions which induce polypeptide expression.

Although expression of the polypeptides of the invention may take place in a Staphylococcus, the invention will usually use a heterologous host for expression (recombinant expression). The heterologous host may be prokaryotic (e.g., a bacterium) or eukaryotic. The heterologous host may be E. coli, but other suitable hosts include Bacillus subtilis, Vibrio cholerae, Salmonella typhi, Salmonella typhimurium, Neisseria lactamica, Neisseria cinerea, Mycobacteria (e.g., M. tuberculosis), yeasts, insect cells, etc. Compared to the wild-type S. aureus genes encoding certain polypeptides of the invention, it may be helpful to change codons to optimize expression efficiency in such hosts without affecting the encoded amino acids. Methods and procedures for codon optimization in target host cells are known in the art.

In an embodiment, the disclosure provides a method for increasing the yield of a polypeptide comprising a modified SACOL1867 polypeptide or fragment thereof by at least 5% (e.g., by at least 5%, 10%, 15%, 20%, 25%, 30%, 40%, 50%, 75%, 100%, 200%, 300%, 400%, 500%, 750%, and up to 1000%) as compared to a polypeptide comprising the SACOL1867 polypeptide of SEQ ID NO:2, the method comprising expressing the polypeptide comprising a modified SACOL1867 polypeptide or fragment thereof in a heterologous host, wherein the yield of the polypeptide comprising a modified SACOL1867 polypeptide or fragment thereof is increased by at least 5% (e.g., by at least 5%, 10%, 15%, 20%, 25%, 30%, 40%, 50%, 75%, 100%, 200%, 300%, 400%, 500%, 750%, and up to 1000%) as compared to a polypeptide comprising the SACOL1867 polypeptide of SEQ ID NO:2. In some embodiments, the yield is measured in soluble fractions of the heterologous host expressing the polypeptide comprising a modified SACOL1867 polypeptide or fragment thereof. In other embodiments, the polypeptide comprising a modified SACOL1867 polypeptide or fragment thereof is secreted from the heterologous host and the yield is measured in the culture media in which the heterologous host was cultured.

The invention also provides a process for producing a polypeptide of the invention comprising the step of synthesizing at least part of the polypeptide by chemical means.

Nucleic Acids

Polynucleotides encoding the modified SACOL1867 polypeptides of the invention, fusions thereof, and/or other antigens used in combination with the modified SACOL1867 polypeptides of the invention are also provided.

The nucleic acids of the present invention preferably comprise a nucleotide sequence encoding one or more proteins/polypeptides described herein (or fragments or variants thereof). In some embodiments, the nucleotide sequence encoding one or more proteins/polypeptides described herein (or fragments or variants thereof) are operably linked to one or more regulatory elements for gene expression, such as, for example, a promoter, an initiation codon, a stop codon, one or more enhancers, and/or a polyadenylation signal. Regulatory elements are preferably selected that are operable in the species to which they are to be administered. In some embodiments, the nucleic acid of the invention is one or more of the nucleotide sequences of SEQ ID NOS: 1 and 84-94.

The invention also provides nucleic acids comprising nucleotide sequences having sequence identity to such nucleotide sequences. Identity between sequences can be determined using any known methods, such as those described above. Such nucleic acids include those using alternative codons to encode the same amino acid.

The invention also provides nucleic acids which can hybridize to these nucleic acids. Hybridization reactions can be performed under conditions of different “stringency”. Conditions that increase stringency of a hybridization reaction are widely known. Examples of relevant conditions include (in order of increasing stringency): incubation temperatures of 25° C., 37° C., 50° C., 55° C., and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art.

In some embodiments, nucleic acids of the invention hybridize to a target under low stringency conditions; in other embodiments, they hybridize under intermediate stringency conditions; in preferred embodiments, they hybridize under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

The invention includes nucleic acids comprising nucleotide sequences complementary to these nucleotide sequences (e.g., for antisense or probing, or for use as primers).

Nucleic acids of the invention can be used in hybridization reactions (e.g., Northern or Southern blots, or in nucleic acid microarrays or “gene chips”) and amplification reactions (e.g., PCR, SDA, SSSR, LCR, TMA, NASBA, etc.) and other nucleic acid techniques.

Nucleic acids according to the invention can take various forms (e.g., single-stranded, double-stranded, vectors, primers, probes, labelled, etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form, i.e., substantially free from other nucleic acids (e.g., free from naturally-occurring nucleic acids), particularly from other staphylococcal or host cell nucleic acids, generally being at least about 50% pure (by weight), and usually at least about 90% pure.

Nucleic acids of the invention may be prepared in many ways, e.g., by chemical synthesis (e.g., phosphoramidite synthesis of DNA) in whole or in part, by digesting longer nucleic acids using nucleases (e.g., restriction enzymes), by joining shorter nucleic acids or nucleotides (e.g., using ligases or polymerases), from genomic or cDNA libraries, etc.

Nucleic acids of the invention may be attached to a solid support (e.g., a bead, plate, filter, film, slide, microarray support, resin, etc.). Nucleic acids of the invention may be labelled, e.g., with a radioactive or fluorescent label, or a biotin label. This is particularly useful where the nucleic acid is to be used in detection techniques, e.g., where the nucleic acid is a primer or as a probe.

The term “nucleic acid” includes a polymeric form of nucleotides of any length, which contain deoxyribonucleotides, ribonucleotides, and/or their analogs. It includes DNA, RNA, and DNA/RNA hybrids. It also includes DNA or RNA analogs, such as those containing modified backbones (e.g., peptide nucleic acids (PNAs) or phosphorothioates) or modified bases. Thus, the invention includes mRNA, tRNA, rRNA, ribozymes, DNA, cDNA, recombinant nucleic acids, branched nucleic acids, plasmids, vectors, probes, primers, etc. Where the nucleic acid of the invention takes the form of RNA, it may or may not have a 5′ cap.

Nucleic acids of the invention may be “naked” or may be part of a vector, i.e., part of a nucleic acid construct designed for transduction/transfection of one or more cell types.

Within the context of the present invention is the in vivo administration of a nucleic acid of the invention to a mammal so that one or more proteins/polypeptides (or a fragment thereof) of interest is/are expressed in the mammal (e.g., nucleic acid vaccine, DNA or RNA vaccine).

Vectors

Vectors comprising polynucleotide sequences encoding modified SACOL1867 polypeptides, fusions thereof, and/or other contemplated compositions are also provided.

Vectors may be, for example, “cloning vectors” which are designed for isolation, propagation and replication of inserted nucleotides, “expression vectors” which are designed for expression of a nucleotide sequence in a host cell, “viral vectors” which are designed to result in the production of a recombinant virus or virus-like particle, or “shuttle vectors”, which comprise the attributes of more than one type of vector. Preferred vectors are plasmids.

Nucleic acids may be delivered to cells in vivo using methods well known in the art such as direct injection of DNA, receptor-mediated DNA uptake, viral-mediated transfection or non-viral transfection and lipid-based transfection, all of which may involve the use of vectors. Direct injection has been used to introduce naked DNA into cells in vivo (see, e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see, e.g., Wu, G. and Wu, C. H. (1988) J. Biol. Chem. 263: 14621; Wilson et al. (1992) J. Biol. Chem. 267: 963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see, e.g., Curiel et al. (1991) Proc. Natl. Acad. Sci. USA 88: 8850; Cristiano et al. (1993) Proc. Natl. Acad. Sci. USA 90:2122-2126).

Useful delivery vectors include biodegradable microcapsules, immuno-stimulating complexes (ISCOMs) or liposomes, and genetically engineered attenuated live vectors such as cells, viruses or bacteria.

Liposome vectors are unilamellar or multilamellar vesicles, having a membrane portion formed of lipophilic material and an interior aqueous portion. The aqueous portion is used in the present invention to contain the polynucleotide material to be delivered to the target cell. It is generally preferred that the liposome forming materials have a cationic group, such as a quaternary ammonium group, and one or more lipophilic groups, such as saturated or unsaturated alkyl groups having about 6 to about 30 carbon atoms. One group of suitable materials is described in European Patent Publication No. 0187702, and further discussed in U.S. Pat. No. 6,228,844 to Wolff et al., the pertinent disclosures of which are incorporated by reference. Many other suitable liposome-forming cationic lipid compounds are described in the literature (see, e.g., L. Stamatatos, et al., Biochemistry 27:3917 3925 (1988); and H. Eibl, et al., Biophysical Chemistry 10:261 271 (1979). Alternatively, a microsphere such as a polylactide-coglycolide biodegradable microsphere can be utilized. A nucleic acid construct is encapsulated or otherwise complexed with the liposome or microsphere for delivery of the nucleic acid to a tissue, as is known in the art.

Preferred viral vectors include Bacteriophages, Herpes virus, Adenovirus, Polio virus, Vaccinia virus, defective retroviruses, adeno-associated virus (AAV) and Avipox. Methods of transforming viral vectors with an exogenous DNA construct are also well described in the art. See Sambrook and Russell, above.

In some embodiments, the nucleic acids of the invention can be incorporated into expression vectors useful for recombinant production of one or more polypeptides of the invention. Suitable expression vectors can be chosen based upon the recombinant system to be used. For example, the pKA80 expression vector can be used for recombinant expression of one or more polypeptides of the invention in E. coli.

Host Cells

Host cells comprising one or more nucleic acids and/or vectors described herein are also provided.

A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

The host cell may be prokaryotic (e.g., a bacterium) or eukaryotic. It may be E. coli, but other suitable host cells include Bacillus subtilis, Vibrio cholerae, Salmonella typhi, Salmonella typhimurium, Neisseria lactamica, Neisseria cinerea, Mycobacteria (e.g., M. tuberculosis), yeasts, etc. In some embodiments, the host cell is a bacterial cell. In a preferred embodiment, the host cell is E. coli.

As indicated above, the nucleic acids of the invention (e.g., DNA or RNA) may be incorporated into a host, such as a host cell, in vitro or ex vivo (e.g., an immune cell, such as a dendritic cell), or in an attenuated microbial host (e.g., attenuated S. aureus, SCV, etc.) by transfection or transformation, and the transfected or transformed cell or microorganism, which expresses the polypeptide (e.g., a modified SACOL1867 polypeptide of the invention alone, or fused to one or more other antigens and/or combined with one or more other antigens), may be administered to the subject. Following administration, the cell will express the protein or polypeptide of interest (or a variant or fragment thereof) in the subject, which will in turn lead to the induction of an immune response directed against the protein, polypeptide or fragment thereof.

The use of attenuated live bacteria to immunize and/or to deliver specific constructs or antigen mixture of the present invention represents an interesting approach to improve immune responses. Live attenuated organisms that mimic natural infection stimulate the immune system in a powerful manner, eliciting broad and robust immune responses that produce both serum and mucosal antibodies, and effector and memory T cells which act synergistically to protect against disease. Examples of suitable attenuated live bacterial vectors include S. aureus, Salmonella typhimurium, Salmonella typhi, Shigella, Bacillus, Lactobacillus, Bacille Calmette-Guerin (BCG), Escherichia coli, Vibrio cholerae, Campylobacter, or any other suitable bacterial vector, as is known in the art. Methods of transforming live bacterial vectors with an exogenous DNA construct are well described in the art. See, for example, Joseph Sambrook and David W. Russell, Molecular Cloning, A Laboratory Manual, 3rd Ed., Cold Spring Harbor Laboratory Press, Cold Spring Harbor, N.Y. (2001). The present invention encompasses the use of a composition comprising an attenuated live bacterium (e.g., ΔhemBΔ720 S. aureus expressing the construct of the present invention as the sole immunogenic component or in combination with other attenuated live bacteria each expressing another polypeptide, fragment or variant described herein (e.g., SACOL0029, SACOL0442, SACOL0720 or fragments or variants thereof).

Compositions

The polypeptides, nucleic acids and delivery systems (e.g., host cells comprising said nucleic acids or vectors) described herein can be formulated into compositions. As used herein, the term “pharmaceutically acceptable” refers to vaccine components (e.g., excipients, carriers, adjuvants) and compositions that are physiologically tolerable and do not typically produce an allergic or similar untoward reaction, such as gastric upset, dizziness and the like, when administered to a subject. Preferably, as used herein, the term “pharmaceutically acceptable” means approved by regulatory agency of the federal or state government or listed in the U.S. Pharmacopeia or other generally recognized pharmacopeia for use in animals, and in humans. The term “excipient” refers to a diluent, carrier, or vehicle with which the vaccine components of the present invention may be administered. Sterile water or aqueous saline solutions and aqueous dextrose and glycerol solutions may be employed as carriers, particularly for injectable solutions.

As used herein, the term “vaccine” refers to any compound/agent (“vaccine component”), or combinations thereof, capable of inducing/eliciting an immune response in a host and which permits treatment and/or prevention of an infection and/or a disease. Therefore, non-limiting examples of such agent include proteins, polypeptides, protein/polypeptide fragments, immunogens, antigens, peptide epitopes, epitopes, mixtures of proteins, peptides or epitopes as well as nucleic acids, genes or portions of genes (encoding a polypeptide or protein of interest or a fragment thereof) added separately or in a contiguous sequence, such as in nucleic acid vaccines, and the like.

In some embodiments, the composition of the present invention is administered in combination with an adjuvant or immunostimulant. Suitable adjuvants or immunostimulants that may improve the efficacy of components to raise an immune response include, but are not limited to, oils (e.g., mineral oils, emulsified oil such as MONTANIDE™ or EMULSIGEN™-D), metallic salts (e.g., alum, aluminum hydroxide, or aluminum phosphate), cationic peptides, such as indolicidin or a cationic peptide produced by the cow's immune cells, natural and artificial microbial components (e.g., bacterial liposaccharides, Freund's adjuvants, muramyl dipeptide (MDP), cyclic-diguanosine-5′-monophosphate (c-di-GMP), pathogen-associated molecular patterns (PAMPS) such as surface polysaccharides, lipopolysaccharides, glycans, peptidoglycan or microbial DNA (e.g., CpG), plant components such as saponins (e.g., Quil-A™), and/or one or more substances that have a carrier effect (e.g., bentonite, latex particles, liposomes, ISCOM™, DNA and polyphosphazine (PCPP) copolymers). Immunization with synthetic nanoparticles (such as those made from a biodegradable synthetic polymer like poly(D,L-lactic co-glycolic acid)) containing antigens plus ligands that signal through TLR to stimulate proinflammatory cytokines is also possible.

Vaccine components of the invention may be administered in a pharmaceutical composition. Pharmaceutical compositions may be administered in unit dosage form. Any appropriate route of administration may be employed, for example, parenteral, subcutaneous, intramuscular, intramammary, intracranial, intraorbital, ophthalmic, intraventricular, intracapsular, intraarticular, intraspinal, intracistemal, intraperitoneal, intranasal, aerosol, or oral administration. Examples of specific routes of administration include parenteral, e.g., intravenous, intradermal, subcutaneous, intramammary; oral (e.g., inhalation); transdermal (topical); transmucosal, and rectal administration.

Conventional pharmaceutical practice may be employed to provide suitable formulations or compositions to administer such vaccine components with or without adjuvants to subjects. Methods well known in the art for making pharmaceutical compositions and formulations are found in, for example, Remington: The Science and Practice of Pharmacy, (20th ed.) ed. A. R. Gennaro A R., 2000, Lippincott: Philadelphia. Formulations for parenteral administration may, for example, contain excipients, sterile water, or saline, polyalkylene glycols such as polyethylene glycol, miglyol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds of the invention include ethylenevinyl acetate copolymer particles, osmotic pumps, implantable infusion systems, and liposomes. Formulations for inhalation or intramammary injection may contain excipients, for example, lactose, or may be aqueous solutions containing, for example, polyoxyethylene-9-lauryl ether, miglyol, glycocholate and deoxycholate, or may be oily solutions (e.g., paraffin oil) for administration in the form of nasal drops, or as a gel.

Therapeutic formulations may be in the form of liquid solutions or suspension; for oral administration, formulations may be in the form of tablets or capsules; and for intranasal formulations, in the form of powders, nasal drops, or aerosols. Solutions or suspensions used for parenteral, intradermal, intramammary or subcutaneous application can include the following components: a sterile diluent such as water for injection, saline solution, fixed oils (e.g., paraffin oil), polyethylene glycols, glycerin, propylene glycol, miglyol or other synthetic solvents; antibacterial agents such as benzyl alcohol or methyl parabens; antioxidants such as ascorbic acid or sodium bisulfite; chelating agents such as ethylenediaminetetraacetic acid; reducing agents such dithiothreitol, buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous or intramammary administration, suitable carriers include physiological saline, bacteriostatic water, Cremophor™ EL™ (BASF, Parsippany, N.J.) or phosphate buffered saline (PBS).

Oral compositions generally include an inert diluent or an edible carrier. They can be enclosed in gelatin capsules or compressed into tablets or feed. For the purpose of oral vaccine administration, the active components can be incorporated with excipients and used in the form of tablets, troches, capsules or in feed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the vaccine components are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Liposomal suspensions (including liposomes targeted to specific cell types) can also be used as pharmaceutically acceptable carriers.

The pharmaceutical compositions may also contain preserving agents, solubilizing agents, stabilizing agents, wetting agents, emulsifiers, sweeteners, colorants, odorants, salts for the variation of osmotic pressure, buffers, coating agents or antioxidants. They may also contain other therapeutically valuable agents.

Intravenous, intramuscular, subcutaneous, intramammary or oral administration is a preferred form of use. The dosages in which the components of the present invention are administered in effective amounts depend on the nature of the specific active ingredient, the host and the requirements of the subject and the mode of application.

Microbial Targets

Polypeptides, nucleic acids and delivery systems of the present invention may be used as antimicrobial agents against Staphylococcal infections including those causing intramammary infection. In a preferred embodiment, the Staphylococcal infections are caused by Staphylococcus aureus.

Methods of Immunization with Polypeptides, Nucleic Acids, Vectors, Cells, Compositions and Delivery Systems

Encompassed by the methods, uses, pharmaceutical compositions and kits of the present invention is passive and active immunization. Passive immunization is the injection of antibodies or antiserum, previously generated against the pathogen (or antigens described herein), in order to protect or cure a recipient animal of an infection or future infection. Protection fades over the course of a few weeks during which time the active immunization with polypeptides, nucleic acids or delivery systems (e.g., as described above) will have time to generate a lasting protective response. Serum for passive immunization can be generated by immunization of donor animals using the polypeptides, nucleic acids or delivery systems, as described herein. This serum, which contains antibodies against the antigens, can be used immediately or stored under appropriate conditions. It can be used to combat acute infections (e.g., intramammary infection) or as a prophylactic. Use of antibodies or serums in a passive immunization can be combined with other agents such as an antibiotic to increase the cure rate of an infection currently in progress or to increase protection against an imminent infection.

Active immunization is administration of the polypeptides, nucleic acids or delivery systems as described herein to a subject.

The components identified in accordance with the teachings of the present invention have a prophylactic and/or therapeutic value such as they can be used to raise an immune response to prevent and/or combat diseases or conditions, and more particularly diseases or conditions related to microbial infections.

The terms “prevent/preventing/prevention” or “treat/treating/treatment” as used herein, refer to eliciting the desired biological response, i.e., a prophylactic and therapeutic effect, respectively, in a subject. In accordance with the present invention, the therapeutic effect comprises one or more of a decrease/reduction in the severity, intensity and/or duration of the microbial infection (e.g., staphylococcal infection) or any symptom thereof following administration of the polypeptide, nucleic acid or delivery system (agent/composition of the present invention) of the present invention when compared to its severity, intensity and/or duration in the subject prior to treatment or as compared to that/those in a non-treated control subject having the infection or any symptom thereof. In accordance with the invention, a prophylactic effect may comprise a delay in the onset of the microbial infection (e.g., staphylococcal infection) or any symptom thereof in an asymptomatic subject at risk of experiencing the microbial infection (e.g., staphylococcal infection) or any symptom thereof at a future time; or a decrease/reduction in the severity, intensity and/or duration of a microbial infection (e.g., staphylococcal infection) or any symptom thereof occurring following administration of the agent/composition of the present invention, when compared to the timing of their onset or their severity, intensity and/or duration in a non-treated control subject (i.e., asymptomatic subject at risk of experiencing the microbial (e.g., bacterial) infection (e.g., staphylococcal infection) or any symptom thereof; and/or a decrease/reduction in the progression of any preexisting microbial infection (e.g., staphylococcal infection) or any symptom thereof in a subject following administration of the agent/composition of the present invention when compared to the progression of microbial infection (e.g., staphylococcal infection) or any symptom thereof in a non-treated control subject having such preexisting microbial infection (e.g., staphylococcal infection) or any symptom thereof. As used herein, in a therapeutic treatment, the agent/composition of the present invention is administered after the onset of the microbial infection (e.g., staphylococcal infection) or any symptom thereof. As used herein, in a prophylactic treatment, the agent/composition of the present invention is administered before the onset of the microbial infection (e.g., staphylococcal infection) or any symptom thereof or after the onset thereof but before the progression thereof.

As used herein, “decrease” or “reduction” of microbial infection (e.g., staphylococcal infection) or any symptom thereof refers to a reduction in a symptom of at least 10% as compared to a control subject (a subject not treated with the agent/composition present invention), in an embodiment of at least 20% lower, in a further embodiment of at least 30% lower, in a further embodiment of at least 40% lower, in a further embodiment of at least 50% lower, in a further embodiment of at least 60% lower, in a further embodiment of at least 70% lower, in a further embodiment of at least 80% lower, in a further embodiment of at least 90% lower, in a further embodiment of 100% lower (complete inhibition).

As used herein, the term “symptom” in reference to a staphylococcal infection refers to any staphylococcal infection symptom such as pain, inflammation, fever, vomiting, diarrhea, fatigue muscle aches, anorexia, dehydration, low blood pressure, cellulitis, impetigo, boil and scalded skin syndrome. More particularly, in reference to a staphylococcal intramammary infection, a staphylococcal intramammary infection symptom refers for example to visual abnormalities in milk (e.g., such as a watery appearance, flakes, clots, malodourous, presence of blood), redness of the udder, swelling in the udder, tenderness in the udder, elevated rectal temperature (>39.0° C.), anorexia, decreased rumen motility and fatigue. An increase in milk somatic cell counts (SCC) is another staphylococcal intramammary infection symptom. Milk somatic cells include white blood cells such as leukocytes or neutrophils as well as epithelial cells. It is generally agreed that a SCC of >200,000/mL may represent a staphylococcal intramammary infection symptom or is indicative of a staphylococcal intramammary infection.

Dosage

Toxicity or efficacy of vaccine components to elicit an immune response can be determined by standard procedures in cell cultures or experimental animals. The dose ratio between toxic and immune stimulatory effects can be measured. Components that exhibit large ratios are preferred. While components that exhibit toxic side effects may be used, care should be taken to design a delivery system in order to minimize potential damage to cells and, thereby, reduce side effects.

Data obtained from cell culture assays and laboratory animal studies can be used in formulating a range of dosage for use in large animals and humans. The dosage of such components lies preferably within a range of administered concentrations that include efficacy with little or no toxicity. The dosage may vary within this range depending upon the dosage form employed and the route of administration utilized.

Any suitable amount of the pharmaceutical composition may be administered to a subject. The dosages will depend on many factors. Typically, the amount of active ingredient contained within a single dose will be an amount that effectively prevents, or treats intramammary infection without inducing significant toxicity. The skilled artisan will appreciate that certain factors may influence the dosage required to effectively raise an immune response in a subject. Moreover, the therapeutically effective amount of the antigens (e.g., modified SACOL1867 polypeptide or fusion polypeptide comprising a modified SACOL1867 polypeptide) of the present invention may require a series of doses. In general, an amount of about 0.01 mg-500 mg of antigens per dose, come into consideration. In some embodiments, an amount of about 0.1 mg-1 mg of antigens per dose, come into consideration. Generally, one, two, or three doses of the vaccine may favor optimal development of immunity. The time between two doses may be as short as three or four weeks but it may be preferred to separate the priming dose (first dose) and the booster dose (second dose) by five, six, seven, eight, nine or ten weeks before stimulating the immune system with the booster shot. A subsequent booster shot (a recall shot) may also be optimal to provide a sustainable immunity. This recall could for example occur every half year (6 months), yearly, every two years, every three or every five years.

“Sample” or “biological sample” refers to any solid or liquid sample isolated from a live being. In a particular embodiment, it refers to any solid (e.g., tissue sample) or liquid sample isolated from a mammal, such as milk, a biopsy material (e.g., solid tissue sample), blood (e.g., plasma, serum or whole blood), saliva, synovial fluid, urine, amniotic fluid and cerebrospinal fluid. Such sample may be, for example, fresh, fixed (e.g., formalin-, alcohol- or acetone-fixed), paraffin-embedded or frozen prior to analysis of the infectious agent's expression level.

Patients

As used herein, the term “subject” or “patient” refers to an animal, preferably a mammal, including, but not limited to, a human, cow (e.g., heifer, multiparous, primiparous, calf), goat, sheep, ewe, ass, horse, pig, chicken, cat, dog, etc. who is the object of treatment, observation, or experiment. In a specific embodiment, the “subject” or “patient” is a cow (e.g., at risk of experiencing staphylococcal infection, such as an intramammary infection).

As used herein, the terms “subject at risk of experiencing a staphylococcal infection (e.g., staphylococcal intramammary infection) or any symptom thereof at a future time” refers to a mammal (e.g., a cow (e.g., heifer, multiparous, primiparous, calf), goat, sheep) that is used for milk or meat production.

In some embodiments, the above-mentioned mammal is a cow.

Method of Detection

Examples of methods to measure the amount/level of selected proteins/polypeptides include, but are not limited to: Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, flow cytometry, and assays based on a property of the protein including, but not limited to, DNA binding, ligand binding, interaction with other protein partners, or enzymatic activity.

In some embodiments, the amount of the polypeptide/protein within the methods of the present invention is detected using antibodies that are directed specifically against the polypeptide/protein. The term “antibody,” as used herein, encompasses monoclonal antibodies, polyclonal antibodies, multispecific antibodies (e.g., bispecific antibodies), and antibody fragments, as long as they exhibit the desired biological activity or specificity. “Antibody fragments” comprise a portion of a full-length antibody, generally the antigen binding or variable region thereof. Interactions between antibodies and a target polypeptide are detected by radiometric, colorimetric, or fluorometric means. Detection of antigen-antibody complexes may be accomplished by addition of a secondary antibody that is coupled to a detectable tag, such as, for example, an enzyme, fluorophore, or chromophore.

Methods for making antibodies are well known in the art. Polyclonal antibodies can be prepared by immunizing a suitable subject (e.g., rabbit, goat, mouse, or other mammal) with the polypeptide/protein of interest or a fragment thereof as an immunogen. A polypeptide/protein “fragment,” “portion,” or “segment” is a stretch of amino acid residues of at least about 5, 7, 10, 14, 15, 20, 21 or more amino acids of the polypeptide noted above. The antibody titer in the immunized subject can be monitored over time by standard techniques, such as with an enzyme linked immunosorbent assay (ELISA) using immobilized exosomal marker polypeptide or a fragment thereof. At an appropriate time after immunization, e.g., when the antibody titers are highest, antibody-producing cells can be obtained from the animal, usually a mouse, and can be used to prepare monoclonal antibodies by standard techniques, such as the hybridoma technique originally described by Kohler and Milstein (1975) Nature 256: 495-497, the human B cell hybridoma technique (Kozbor et al. (1983) Immunol. Today 4: 72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology, John Wiley & Sons, Inc., New York, N.Y.).

Alternatively, to preparing monoclonal antibody-secreting hybridomas, a monoclonal antibody can be identified and isolated by screening a recombinant combinatorial immunoglobulin library (e.g., an antibody phage display library) with a polypeptide or a fragment thereof to thereby isolate immunoglobulin library members that bind the polypeptide. Kits for generating and screening phage display libraries are commercially available (e.g., the Pharmacia Recombinant Phage Antibody System™, Catalog No. 27-9400-01; and the Stratagene SurfZAP™ Phage Display Kit, Catalog No. 240612).

Furthermore, antibodies directed against one or more of the polypeptides/proteins described herein may be obtained from commercial sources.

The use of immobilized antibodies specific for the polypeptides/proteins is also contemplated by the present invention and is well known by one of ordinary skill in the art. The antibodies could be immobilized onto a variety of solid supports, such as magnetic or chromatographic matrix particles, the surface of an assay plate (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality (2 or more) of polypeptides/proteins may be carried out separately or simultaneously with one test sample. Several polypeptides/proteins may be combined into one test for efficient processing of a multiple of samples.

The analysis of polypeptides/proteins could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340, 2002) and capillary devices.

In an embodiment, the above-mentioned level of expression is determined by measuring the level of expression of an mRNA transcribed from said one or more genes.

Methods to determine nucleic acid (e.g., mRNA) levels are known in the art, and include for example polymerase chain reaction (PCR), reverse transcriptase-PCR (RT-PCR), SAGE, quantitative PCR (q-PCR), Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the nucleic acids encoding the protein/polypeptide of this invention; amplification of mRNA expressed from one or more of the nucleic acids encoding the proteins/polypeptides of this invention using specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the biological sample, which is then labeled and used to probe cDNAs or oligonucleotides encoding all or part of the nucleic acids encoding the proteins/polypeptides of this invention, arrayed on any of a variety of surfaces.

Kits

The present invention also encompasses kits comprising the component(s) of the present invention. For example, the kit can comprise one or more components. The components can be packaged in a suitable container and device for administration. The kit can further comprise instructions for using the kit.

The present invention also provides a kit or package comprising reagents useful for administering one or more polypeptide, fusion polypeptide, nucleic acid, vector, host, compositions of the present invention, or a combination of at least two thereof, to a subject in need thereof for treating and/or preventing Staphylococcal infection, such as Staphylococcal intramammary infection. Such kit may further comprise, for example, instructions for the prevention and/or treatment of Staphylococcal infection (e.g., Staphylococcal intramammary infection), containers, and/or reagents useful for performing the methods. The kit may further include, where necessary, agents for reducing background interference in a test, agents for increasing signal, software and algorithms for combining and interpolating marker values to produce a prediction of clinical outcome of interest, apparatus for conducting a test, calibration curves and charts, standardization curves and charts, and the like.

### EXAMPLES

### Example 1

Mutation of SACOL1867 to Improve Yield and Stability During Recombinant Expression

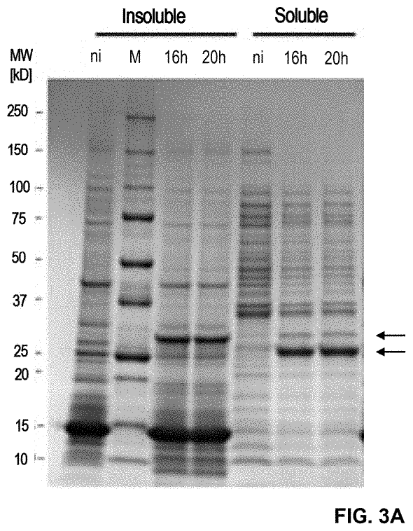

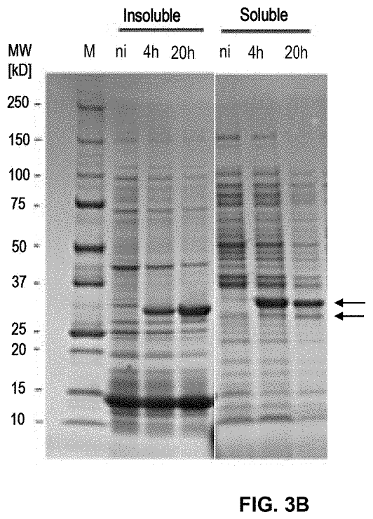

While upscaling the production of fusion antigen, SACOL0029-1867 (SEQ ID NO:80), unexpected problems were noted during the purification of recombinant SACOL0029-1867 expressed from Escherichia (E.) coli. As shown in FIG. 3A, while ample amounts of recombinant SACOL0029-1867 product could be detected in the E. coli biomass after IPTG induction, only a minor fraction could be purified by affinity of the N-terminally added hexa-histidine (Hiss) tag to Ni²⁺-NTA or Ni²⁺-IDA resins, leading to low yields. The unbound SACOL0029-1867 fraction also possessed higher mobility on SDS-polyacrylamide gels, indicative of a shorter polypeptide chain compared to the intended expression product. The upper arrow of FIG. 3A indicates the expected size of SACOL0029-1867 and the lower arrow indicates the actual size of the SACOL0029-1867 polypeptide that was obtained. Based on the appearance of the SACOL0029-1867 expression products, it was hypothesized that a proteolytic degradation problem had occurred. Both identified issues (low yields and questionable identity) posed significant problems for potential vaccine development.

Some of the conventional solutions when encountering such a recombinant protein stability problem, such as lower expression temperature, variations of growth medium (salts, nutrients), shortening or variation of linker sequences with predicted exposed secondary structure and not directly belonging to the intended recombinant protein were attempted without success, i.e., did not sufficiently suppress the proteolytic degradation. This cast doubt on the conventional notion that endogenous E. coli proteases were the root of the problem.

Thus, the antigen to be expressed, SACOL0029-1867, was re-examined. This protein is a fusion between the first 55 amino acids of SACOL0029 (a putative HMG-CoA synthase; SEQ ID NO:19) and the mature protein (lacking the secretory signal sequence) of SACOL1867 (SEQ ID NO:3). The latter has been identified as a S. aureus protein (SpIC), belonging to the serine protease operon spl (Reed et al., Infect Immunol., 2001, 69(3):1521-7) encoding six serine protease-like proteins (SplA-SplF). A crystal structure has been described for SpIC and its fold has been described as resembling the one known from the V8 protease (Popowicz et al., J Mol Biol., 2006, 358(1):270-9). However, while for other members of the Spl operon, proteolytic activity could be shown (e.g., Reed et al., Infect Immunol., 2001, 69(3):1521-7, Popowicz et al., J Mol Biol., 2006, 358(1):270-9, Zdzalik et al., PLoS One, 2013, 8(10):e76812, Stach et al., Structure, 2018, 26(4):572-579), no such activity could be demonstrated for SpIC, despite the availability of folded recombinant protein (Popowicz et al., J Mol Biol., 2006, 358(1):270-9). Internal efforts to demonstrate a proteolytic activity by standard protease assays were also unsuccessful.

However, close inspection of the available sequences and multiple sequence alignments suggested that the catalytic triad in SpIC (SACOL1867) is most likely intact (Reed et al., Infect Immunol., 2001, 69(3):1521-7, Popowicz et al., J Mol Biol., 2006). Therefore, despite the absence of experimental evidence that SACOL1867 possesses protease activity, we generated a SACOL0029-1867 point mutant at the hypothetical active site (corresponding to amino acid position 193 in SEQ ID NO:2) to mutate the serine residue to an alanine residue. By removing the nucleophilic serine hydroxyl moiety, it was reasoned that the mutant protein should be incapable of proteolysis. If proteolytic activity of the SACOL0029-1867 polypeptide was the cause of the stability problem outlined above, stability should be restored in the mutant SACOL0029-1867 polypeptide. E. coli expression experiments with the mutant SACOL0029-1867 construct confirmed the hypothesis. As shown in FIG. 3B, the degradation product (indicated by the lower arrow) obtained from recombinant expression of the wild-type SACOL0029-1867 (SEQ ID NO:80) was absent upon expression of the mutant SACOL0029-1867 (SEQ ID NO:67) and yields obtained upon expression of the mutant SACOL0029-1867 (SEQ ID NO:67) were high.

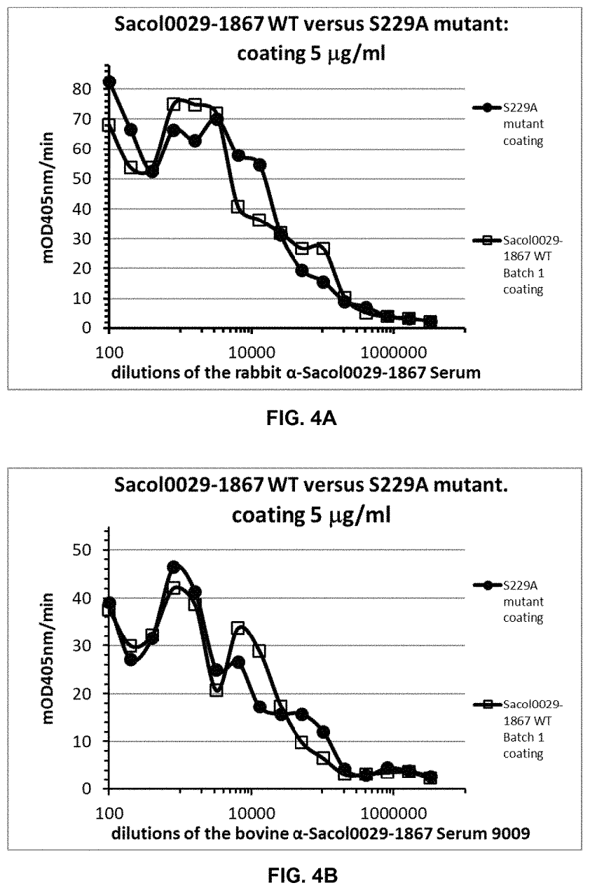

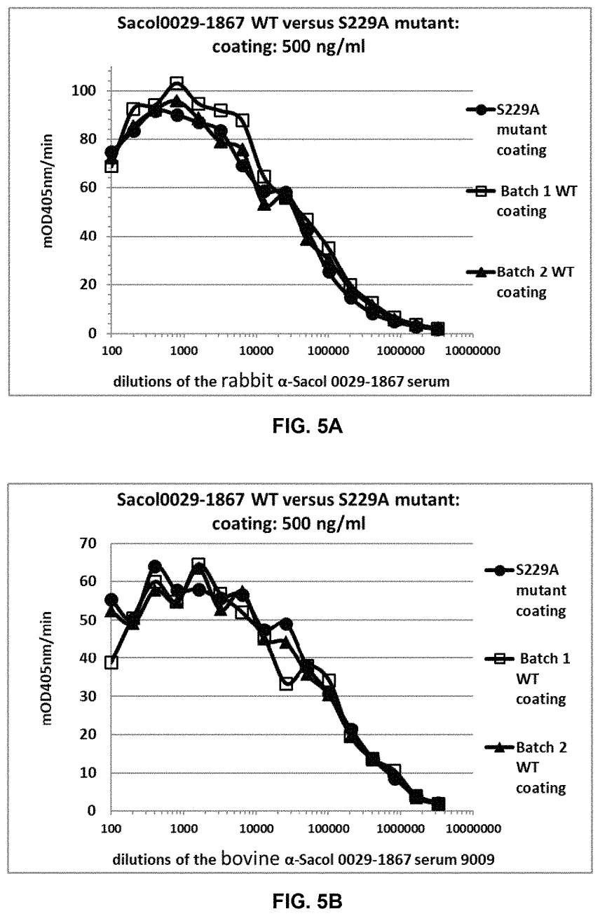

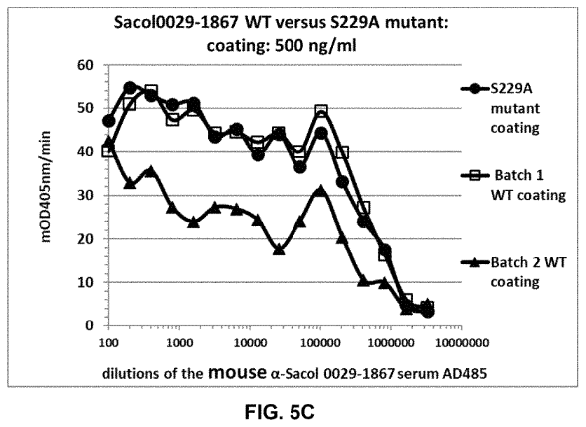

To demonstrate that the mutant SACOL0029-1867 protein (SEQ ID NO:67) has similar or identical immunological properties to the wild-type SACOL0029-1867 (SEQ ID NO:80), comparative indirect ELISA analyses of antisera directed against wild-type SACOL0029-1867 (SEQ ID NO:80) and mutant SACOL0029-1867 (SEQ ID NO:67) were performed under both saturating and sub-saturating antigen coating conditions. No significant differences in ELISA titers could be detected between the wild-type SACOL0029-1867 polypeptide (SEQ ID NO:80) and the mutant SACOL0029-1867 polypeptide (SEQ ID NO:67). Furthermore, no significant differences in curve shape could be detected between the wild-type SACOL0029-1867 polypeptide (SEQ ID NO:80) and the mutant SACOL0029-1867 polypeptide (SEQ ID NO:67) using specific anti-SACOL0029-1867 sera from three different mammalian species (rabbit, bovine, and mouse). This data is illustrated in FIGS. 4A-B and 5A-C. The ELISA data indicate that the S229A point mutation in SACOL0029-1867 does not alter the antigenic properties of this antigen.

This effectiveness of this approach would not have been expected in view of the complexities in antigen expression systems coupled with previous lack of protease activity demonstrated for SACOL1867.

### Example 2

Mutation of SACOL0029-1867 Antigen does not Negatively Affect Antigenicity in Cows

Serum and ELISA results of two groups of cows were compared. The first group of cows were vaccinated with a vaccine formulation containing the wild-type SACOL0029-1867 antigen according to SEQ ID NO: 80 (cows #518, 603, 185, 203, 602, 9007). The second group of cows were vaccinated with a vaccine formulation containing the mutant SACOL0029-1867 antigen according to SEQ ID NO: 67 (cows #637, 228, 636). The adjuvant and vaccination schedule were the same for both groups of animals. The only difference was the utilization of wild-type SACOL0029-1867 antigen (SEQ ID NO: 80) vs. mutant SACOL0029-1867 antigen (SEQ ID NO: 67).

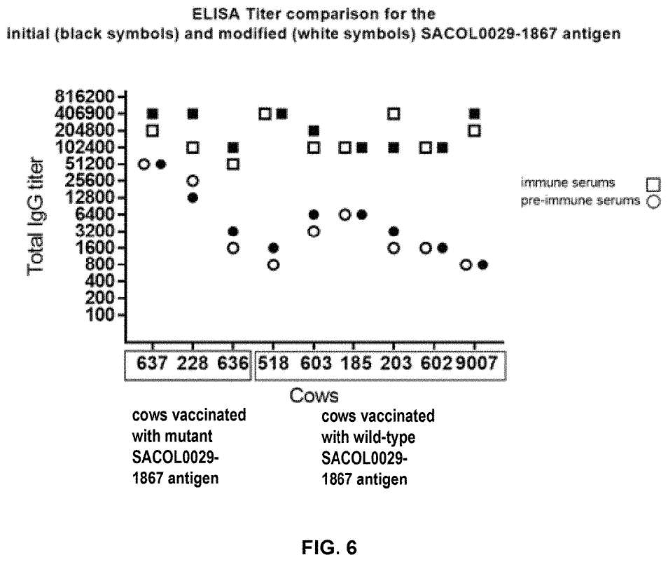

Serums from these two groups of cows were tested on two series of ELISA plates, one series coated with the wild-type SACOL0029-1867 antigen according to SEQ ID NO: 80 (FIG. 6, black symbols), and the other coated with the mutant SACOL0029-1867 antigen according to SEQ ID NO: 67 (FIG. 6, white symbols) to verify that antibodies generated by the vaccine formulation containing the mutant SACOL0029-1867 antigen can recognize the wild-type SACOL0029-1867 antigen and vice versa. Results from the pre-immune serums from both groups of cows (FIG. 6, circles) were compared to serums from vaccinated cows (immune serums 4 weeks after the second immunization [week 14], FIG. 6, squares).

Results from this experiment are shown in FIG. 6 which shows that serums from each cow, regardless of whether the cow was vaccinated with the formulation containing wild-type antigen or the formulation containing mutant antigen, recognized both antigens equally (wild-type SACOL0029-1867 [black symbols] and mutant SACOL0029-1867 [white symbols]). This demonstrates that the mutant SACOL0029-1867 antigen according to SEQ ID NO: 67, which is more stable upon preparation, can be used for vaccination and that antibodies from cows vaccinated with the mutant SACOL002-1867 antigen can recognize the “natural” protein sequence from the pathogen Staphylococcus aureus.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A modified SACOL1867 polypeptide comprising a mutation at one or more amino acid positions, wherein at least one of the mutations disrupts the catalytic binding site and/or decreases proteolytic activity of the modified SACOL1867 polypeptide compared to a reference SACOL1867 polypeptide.

2. The modified SACOL1867 polypeptide of claim 1, wherein at least one of the mutations is at a position corresponding to an amino acid residue within the catalytic triad of the SACOL1867 polypeptide sequence.

3. The modified SACOL1867 polypeptide of claim 1, wherein at least one of the mutations is at a position corresponding to amino acid residue 75, 113, and/or 193 of SEQ ID NO:2.

4. The modified SACOL1867 polypeptide of claim 1, wherein at least one of the mutations is at a position corresponding to amino acid residue 193 of SEQ ID NO:2.

5. The modified SACOL1867 polypeptide of claim 4, wherein the mutation at a position corresponding to amino acid residue 193 of SEQ ID NO:2 is an amino acid substitution to alanine.

6. The modified SACOL1867 polypeptide of claim 1, wherein the modified SACOL1867 polypeptide comprises the amino acid sequence of SEQ ID NO: 5 or 6.

7. A fusion polypeptide of formula I:
X-A-linker-B—Z (formula I),
wherein A and/or B is a modified SACOL1867 polypeptide according to claim 1; the linker is an amino acid sequence of at least one amino acid or is absent; X is an amino acid sequence of at least one amino acid or is absent; and Z is an amino acid sequence of at least one amino acid or is absent.

8. The fusion polypeptide of claim 7,
wherein A is selected from a polypeptide comprising a SACOL0029 polypeptide as set forth in any one of SEQ ID NOS:19-30, a SACOL0264 polypeptide as set forth in SEQ ID NO:31, a SACOL0442 polypeptide as set forth in any one of SEQ ID NOS: 32-43, a SACOL0718 polypeptide as set forth in SEQ ID NO:44, a SACOL0720 polypeptide as set forth in any one of SEQ ID NOS: 45-57, a SACOL1353 polypeptide as set forth in SEQ ID NO:58, a SACOL1416 polypeptide as set forth in SEQ ID NO:59, a SACOL1611 polypeptide as set forth in SEQ ID NO:60, a SACOL1867 polypeptide as set forth in any one of SEQ ID NOS: 2, 3, and 5-18, a SACOL1912 polypeptide as set forth in SEQ ID NO:61, a SACOL1944 polypeptide as set forth in SEQ ID NO:62, a SACOL2144 polypeptide as set forth in SEQ ID NO:63, a SACOL2365 polypeptide as set forth in SEQ ID NO:64, a SACOL2385 polypeptide as set forth in SEQ ID NO:65, or a SACOL2599 polypeptide as set forth in SEQ ID NO:66; and
wherein B is a modified SACOL1867 polypeptide according to claim 1.

9. The fusion polypeptide of claim 7, wherein the linker is an amino acid sequence selected from GGGGSGGGGSGGGGS (SEQ ID NO:69), ERKYK (SEQ ID NO:70), and EAAAKEAAAK (SEQ ID NO:71).

10. The fusion polypeptide of claim 7 comprising the amino acid sequence of SEQ ID NO:67.

11. A nucleic acid comprising a polynucleotide sequence encoding a modified SACOL1867 polypeptide of claim 1 or a fusion polypeptide thereof.

12. A vector comprising the nucleic acid of claim 11.

13. A host cell comprising the vector of claim 12.

14. A method for recombinantly expressing a modified SACOL1867 polypeptide and/or a fusion polypeptide comprising a modified SACOL1867 polypeptide comprising culturing the host cell of claim 13 under conditions conducive for expression of the modified SACOL1867 polypeptide and/or the fusion polypeptide comprising a modified SACOL1867 polypeptide.

15. A composition comprising:
(i) a modified SACOL1867 polypeptide of claim 1 or a fusion polypeptide thereof; and
(ii) a pharmaceutically acceptable excipient and/or an adjuvant.

16. A kit comprising:
(i) a modified SACOL1867 polypeptide of claim 1 or a fusion polypeptide thereof; and
(ii) instructions for using the kit.

17. A method for preventing, treating, and/or controlling a Staphylococcal infection in a subject comprising administering an effective amount of a modified SACOL1867 polypeptide of claim 1 or a fusion polypeptide thereof.

18. A method for improving recombinant expression of a SACOL1867 polypeptide comprising expressing a modified SACOL1867 polypeptide of claim 1 and/or a fusion polypeptide thereof in a host cell, wherein the host cell is cultured under conditions conducive to expression of said modified SACOL1867 polypeptide and/or said fusion polypeptide, and wherein the yield of said modified SACOL1867 polypeptide and/or said fusion polypeptide is increased compared to expression of a polypeptide comprising a wild-type SACOL1867 polypeptide under the same conditions.

19. The method of claim 18, wherein said polypeptide comprising a wild-type SACOL1867 polypeptide is selected from SEQ ID NO: 2, 3, and 80.


SEQUENCE LISTING (94 sequences)

SEQ ID NO: 1 - DNA, 720 bp, Staphylococcus aureus
atgaataaaaatatagtcattaaaagcatggcagcattagccattctaacctcagtaactggaataaatgctgcagtcgttgaagagacacaacaaatagcaaatgcagagaagaatgttacgcaagttaaagatacaaatatttttccatataatggcgtcgtttcatttaaagatgcgacaggttttgtaattggaaaaaatacaattatcaccaataaacatgtatcaaaagattataaagttggcgatagaattactgcccatccaaacggtgacaaaggaaatggtggtatatataaaattaaaagcatttctgattatccgggtgatgaagacatctctgtcatgaatattgaagaacaagcagtcgaacgtggaccaaaaggctttaattttaatgaaaatgtccaagcattcaattttgcgaaagatgctaaagttgatgacaaaattaaagttattggttacccattacctgctcaaaatagttttaaacagtttgaatctacaggaactataaaaagaatcaaagacaatattttaaattttgatgcatacattgaacccgggaattcaggatcaccagttctaaattctaacaatgaggtcataggtgtggtgtatggcggtattggaaaaattggttctgaatataatggtgccgtatactttacgcctcaaatcaaagattttattcaaaagcacattgaacaataa

SEQ ID NO: 2 - protein, 239 aa, Staphylococcus aureus
MNKNIVIKSMAALAILTSVTGINAAVVEETQQIANAEKNVTQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 3 - protein, 199 aa, Staphylococcus aureus
TQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 4 - skipped

SEQ ID NO: 5 - protein, 239 aa, Artificial Sequence
  SACOL1867 mutant
MNKNIVIKSMAALAILTSVTGINAAVVEETQQIANAEKNVTQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNAGSPVLNSNNEVIGVVYGGIGKIGSEYNGAVYFTPQIKDFIQKHIEQ

SEQ ID NO: 6 - protein, 199 aa, Artificial Sequence
  SACOL1867 mutant minus transmembrane domain
TQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNAGSPVLNSNNEVIGVVYGGIGKIGSEYNGAVYFTPQIKDFIQKHIEQ

SEQ ID NO: 7 - protein, 239 aa, Staphylococcus aureus
MNKNIVIKSMAALAILTSVTGINAAVVEETQQIANAEKNVTQVKDTNIFPYNGVVSFKDATGFVIGKNTIITNKHVSKDYKVGDRITAHPDGDKGNGGIYKIKSISDYPGDEDISVMNIEEQAVERGPKGFNFNENVQALNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 8 - protein, 239 aa, Staphylococcus aureus
MNKNIVIKSMAALAILTSATGINAAVVEETQQIANAEKNVTQVKDTNIFPYNGVVSFKDATGFVIGKNTIITNKHVSKDYKVGDRITAHPDGDKGNGGIYKIKSISDYPGDEDISVMNIEEQAVERGPKGFNFNENVQALNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 9 - protein, 239 aa, Staphylococcus aureus; identical to SEQ ID NO: 2

SEQ ID NO: 10 - protein, 239 aa, Staphylococcus aureus; identical to SEQ ID NO: 2

SEQ ID NO: 11 - protein, 239 aa, Staphylococcus aureus; identical to SEQ ID NO: 2

SEQ ID NO: 12 - protein, 239 aa, Staphylococcus aureus
MNKNIVIKSMAALAILTSVTGINAAVVEETQQIANAEKNVTQVKDTNNFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 13 - protein, 239 aa, Staphylococcus aureus; identical to SEQ ID NO: 12

SEQ ID NO: 14 - protein, 239 aa, Staphylococcus aureus; identical to SEQ ID NO: 12

SEQ ID NO: 15 - protein, 239 aa, Staphylococcus aureus; identical to SEQ ID NO: 12

SEQ ID NO: 16 - protein, 239 aa, Staphylococcus aureus; identical to SEQ ID NO: 12

SEQ ID NO: 17 - protein, 239 aa, Staphylococcus aureus
MNKNIVIKSMAALAILTSVTGINAAVVDETQQIANAEKNVTQVKDTNVFPYNGVVSFKDATGFAIEKNTIITNKHVSKDYKVGDRITAHPNGDKGNGGIYKIKNISDYPGNEDISVMNVEENAVERGANGYNFNENVQAFNFAKDAKVDDKIKVIGYPLPAQNTFKQFESTGTVKSIKDNNLNFDAYIEPGNSGSPVLNLNNEVVGVVYGGIGKIGSEYNGAVYFTPQIKEFIQKHIEQ

SEQ ID NO: 18 - protein, 239 aa, Artificial Sequence
  Consensus sequence from SACOL1867 orthologue alignment
  MISC_FEATURE (19)..(19): Xaa = any amino acid
  MISC_FEATURE (28)..(28): Xaa = any amino acid
  MISC_FEATURE (48)..(48): Xaa = any amino acid
  MISC_FEATURE (64)..(64): Xaa = any amino acid
  MISC_FEATURE (66)..(66): Xaa = any amino acid
  MISC_FEATURE (91)..(91): Xaa = any amino acid
  MISC_FEATURE (104)..(104): Xaa = any amino acid
  MISC_FEATURE (111)..(111): Xaa = any amino acid
  MISC_FEATURE (119)..(119): Xaa = any amino acid
  MISC_FEATURE (122)..(122): Xaa = any amino acid
  MISC_FEATURE (128)..(128): Xaa = any amino acid
  MISC_FEATURE (129)..(129): Xaa = any amino acid
  MISC_FEATURE (131)..(131): Xaa = any amino acid
  MISC_FEATURE (140)..(140): Xaa = any amino acid
  MISC_FEATURE (164)..(164): Xaa = any amino acid
  MISC_FEATURE (174)..(174): Xaa = any amino acid
  MISC_FEATURE (176)..(176): Xaa = any amino acid
  MISC_FEATURE (181)..(181): Xaa = any amino acid
  MISC_FEATURE (200)..(200): Xaa = any amino acid
  MISC_FEATURE (205)..(205): Xaa = any amino acid
  MISC_FEATURE (231)..(231): Xaa = any amino acid
MNKNIVIKSMAALAILTSXTGINAAVVXETQQIANAEKNVTQVKDTNXFPYNGVVSFKDATGFXIXKNTIITNKHVSKDYKVGDRITAHPXGDKGNGGIYKIKXISDYPGXEDISVMNXEEXAVERGXXGXNFNENVQAXNFAKDAKVDDKIKVIGYPLPAQNXFKQFESTGTXKXIKDNXLNFDAYIEPGNSGSPVLNXNNEVXGVVYGGIGKIGSEYNGAVYFTPQIKXFIQKHIEQ

SEQ ID NO: 19 - protein, 55 aa, Staphylococcus aureus
MLESREQLSVEEYETFFNRFDNQEFDFERELTQDPYSKVYLYSIEDHIRTYKIEK

SEQ ID NO: 20 - protein, 55 aa, Staphylococcus aureus; identical to SEQ ID NO: 19

SEQ ID NO: 21 - protein, 55 aa, Staphylococcus aureus; identical to SEQ ID NO: 19

SEQ ID NO: 22 - protein, 88 aa, Staphylococcus aureus
CLFSYGSGAVGEIFSGSIVKGYDKALDKEKHLNMLESREQLSVEEYETFFNRFDNQEFDFERELTQDPYSKVYLYSIEDHIRTYKIEK

SEQ ID NO: 23 - protein, 88 aa, Staphylococcus aureus; identical to SEQ ID NO: 22

SEQ ID NO: 24 - protein, 88 aa, Staphylococcus aureus; identical to SEQ ID NO: 22

SEQ ID NO: 25 - protein, 78 aa, Staphylococcus aureus
GEFYSATLVEGYKDHLDQAAHKALLNNRTEVSVDAYETFFKRFDDVDFDEQQDAVHEDRRIFYLSNIENNVREYHRPE

SEQ ID NO: 26 - protein, 88 aa, Staphylococcus aureus
GLFSYGSGSVGEFYSATLVEGYKDHLDQAAHKALLNNRTEVSVDAYETFFKRFDDVDFDEQQDAVHEDRRIFYLSNIENNVREYHRPE

SEQ ID NO: 27 - protein, 78 aa, Staphylococcus aureus
GEFYSATLVEGYKDHLDQAAHKALLNNRTEVSVDAYETFFKRFDDVDFDEEQDAVHEDRHIFYLSNIENNVREYHRPE

SEQ ID NO: 28 - protein, 88 aa, Staphylococcus aureus
GLFSYGSGSVGEFYSATLVEGYKDHLDQAAHKALLNNRTEVSVDAYETFFKRFDDVEFDEEQDAVHEDRHIFYLSNIENNVREYHRPE

SEQ ID NO: 29 - protein, 88 aa, Staphylococcus aureus; identical to SEQ ID NO: 28

SEQ ID NO: 30 - protein, 88 aa, Artificial Sequence
  consensus sequence for SACOL0029 orthologue alignment
  MISC_FEATURE (1)..(34): Xaa = any amino acid
  MISC_FEATURE (36)..(37): Xaa = any amino acid
  MISC_FEATURE (39)..(41): Xaa = any amino acid
  MISC_FEATURE (44)..(45): Xaa = any amino acid
  MISC_FEATURE (51)..(51): Xaa = any amino acid
  MISC_FEATURE (55)..(57): Xaa = any amino acid
  MISC_FEATURE (60)..(72): Xaa = any amino acid
  MISC_FEATURE (75)..(76): Xaa = any amino acid
  MISC_FEATURE (79)..(81): Xaa = any amino acid
  MISC_FEATURE (83)..(83): Xaa = any amino acid
  MISC_FEATURE (85)..(88): Xaa = any amino acid
XXXXXXXXXXXXXXXXXXXXXXXXXXXXXXXXXXLXXRXXXSVXXYETFFXRFDXXXFDXXXXXXXXXXXXXYLXXIEXXXRXYXXXX

SEQ ID NO: 31 - protein, 219 aa, Staphylococcus aureus
MIEINNLSKRYRNKQIFNHLTMSFDSNRLTVLLGDNGAGKSTLLRMIAGIEKANDGTINYFGEKWNQRQIQNHIGYVPQDIALFEHMTVAENIKFFKSLCKNPINDTTINEYLQQLNFDDTSAKVSTLSGGNKRKINILVGLLGQPRILILDEPTVGIDLKSRHDIHQLLNIMKSKCLIILTTHHLDEVEALADDIKLIGQDPFYQHVLEDKQWTYTYY

SEQ ID NO: 32 - protein, 203 aa, Staphylococcus aureus
MFKKYDSKNSIVLKSILSLGIIYGGTFGIYPKADASTQNSSSVQDKQLQKVEEVPNNSEKALVKKLYDRYSKDTINGKSNKSRNWVYSERPLNENQVRIHLEGTYTVAGRVYTPKRNITLNKEVVTLKELDHIIRFAHISYGLYMGEHLPKGNIVINTKDGGKYTLESHKELQKDRENVKINTADIKNVTFKLVKSVNDIEQV

SEQ ID NO: 33 - protein, 203 aa, Staphylococcus aureus; identical to SEQ ID NO: 32

SEQ ID NO: 34 - protein, 203 aa, Staphylococcus aureus; identical to SEQ ID NO: 32

SEQ ID NO: 35 - protein, 203 aa, Staphylococcus aureus; identical to SEQ ID NO: 32

SEQ ID NO: 36 - protein, 203 aa, Staphylococcus aureus
MFKKYDSKNSIVLKSILSLGIIYGGTFGIYPKADASTQNSSSVQDKQLQKVEEVPNNSEKALVKKLYDRYSKDTINGKSNKSRNWVYSERPLNENQVRIHLEGTYTVADRVYTPKRNITLNKEVVTLKELDHIIRFAHISYGLYMGEHLPKGNIVINTKDGGKYTLESHKELQKDRENVKINTADIKNVTFKLVKSVNDIEQV

SEQ ID NO: 37 - protein, 203 aa, Staphylococcus aureus; identical to SEQ ID NO: 36

SEQ ID NO: 38 - protein, 203 aa, Staphylococcus aureus; identical to SEQ ID NO: 36

SEQ ID NO: 39 - protein, 203 aa, Staphylococcus aureus; identical to SEQ ID NO: 36

SEQ ID NO: 40 - protein, 203 aa, Staphylococcus aureus; identical to SEQ ID NO: 36

SEQ ID NO: 41 - protein, 203 aa, Staphylococcus aureus
MFKKNDSKNSILLKSILSLGIIYGGTFGIYPKADASTQNSPSVQDKQFQKVEEVPNNSEKALVKKLYDRYSQNTINGKSNKSRNWVYSERPLNENQVRINLEGTYRVADRVYTPKRNITLNKEVVTLKELDHIIRFAHISYGLYMGEHLPKGNIVINTKDGGKYTLESHKELQKDRENVKINTADIKNVTFKLVKSVNDIEQV

SEQ ID NO: 42 - protein, 203 aa, Staphylococcus aureus
MFKKYDSKNSIVLKSILSLGIIYGGTFGIYPKADASTQNSSSVQDKQFQKVEEVPNNSEKALVKKLYDRYSQNTINGKSNKARNWVYSERPLNENQVRIHLEGTYRVADRVYTPKRNITLNKEVVTLKELDHIIRFAHISYGLYMGEHLPKGNIVINTKNGGKYTLESHKELQKNRENVEINTDDIKNVTFELVKSVNDIEQV

SEQ ID NO: 43 - protein, 203 aa, Artificial Sequence
  consensus sequence from SACOL0442 orthologue alignment
  MISC_FEATURE (5)..(5): Xaa = any amino acid
  MISC_FEATURE (12)..(12): Xaa = any amino acid
  MISC_FEATURE (41)..(41): Xaa = any amino acid
  MISC_FEATURE (48)..(48): Xaa = any amino acid
  MISC_FEATURE (72)..(73): Xaa = any amino acid
  MISC_FEATURE (82)..(82): Xaa = any amino acid
  MISC_FEATURE (100)..(100): Xaa = any amino acid
  MISC_FEATURE (106)..(106): Xaa = any amino acid
  MISC_FEATURE (109)..(109): Xaa = any amino acid
  MISC_FEATURE (160)..(160): Xaa = any amino acid
  MISC_FEATURE (175)..(175): Xaa = any amino acid
  MISC_FEATURE (180)..(180): Xaa = any amino acid
  MISC_FEATURE (184)..(184): Xaa = any amino acid
  MISC_FEATURE (192)..(192): Xaa = any amino acid
  MISC_FEATURE (201)..(203): Xaa = any amino acid
MFKKXDSKNSIXLKSILSLGIIYGGTFGIYPKADASTQNSXSVQDKQXQKVEEVPNNSEKALVKKLYDRYSXXTINGKSNKXRNWVYSERPLNENQVRIXLEGTYXVAXRVYTPKRNITLNKEVVTLKELDHIIRFAHISYGLYMGEHLPKGNIVINTKXGGKYTLESHKELQKXRENVXINTXDIKNVTFXLVKSVNDIXXX

SEQ ID NO: 44 - protein, 253 aa, Staphylococcus aureus
MAILEVKQLTKIYGTKKMAQEVLRDINMSIEEGEFIAIMGPSGSGKTTLLNVLSSIDYISQGSITLKGKKLEKLSNKELSDIRKHDIGFIFQEYNLLHTLTVKENIMLPLTVQKLDKEHMLNRYEKVAEALNILDISDKYPSELSGGQRQRTSAARAFITLPSIIFADEPTGALDSKSTQDLLKRLTRMNEAFKSTIIMVTHDPVAASYANRVVMLKDGQIFTELYQGDDDKHTFFKEIIRVQSVLGGVNYDL

SEQ ID NO: 45 - protein, 629 aa, Staphylococcus aureus
MTFNEIIFKNFRQNLSHYAIYLFSLITSVVLYFSFVALKYAHKLNMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGTLMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKAKNKVDKSIETRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 46 - protein, 629 aa, Staphylococcus aureus; identical to SEQ ID NO: 45

SEQ ID NO: 47 - protein, 599 aa, Staphylococcus aureus
MYFSFVALKYAHKLNMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGTLMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKAKNKVDKSIETRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 48 - protein, 584 aa, Staphylococcus aureus
MTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGTLMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKAKNKVDKSIETRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 49 - protein, 584 aa, Staphylococcus aureus
MTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLIITGYYLSLNIVQYYDSIGILMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKVKNKVDKSIKTRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 50 - protein, 584 aa, Staphylococcus aureus
MTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLIITGYYLSLNIVQYYDSIGILMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKVKNKVDKSIKTRSEAISSISSLTGILLFVTSFLGITFLIAVCCIIYIKQIDETEDELENYSILRKLGFTQKDMARGLKFKIMFNFGLPLVIVLSHAYFTSLAYMKLMGTTNQIPVFIVMGLYICMYAVFAVTAYNHSKRTIRHSI

SEQ ID NO: 51 - protein, 584 aa, Staphylococcus aureus
MTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGILMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKVKNKVDKSIKTRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 52 - protein, 629 aa, Staphylococcus aureus
MTFNEIIFKNFRQNLSHYAIYLFSLITSVVLYFSFVALKYAHKLNMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGILMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAIIGTKKHHVNIKLRKDINKIYFMTDVDLGGPTFVLNDKDYQEIRKYTKAKHIVSQFGFDLKHKKDALALEKVKNKVDKSIKTRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 53 - protein, 629 aa, Staphylococcus aureus; identical to SEQ ID NO: 52

SEQ ID NO: 54 - protein, 629 aa, Staphylococcus aureus
MTFNEIIFKNFRQNLSHYAIYLFSLITSVVLYFSFVALKYAHKLNMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGILMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKAKEPYNVTITSDKYIPNTDLKRGQADLFVAEGSIKDLVKHKKHGKAVIGTKKHHVNIKLRKDINKIYFMTDVDLGGPTFVLNDKDYQEIRKYTKAKHIVSQFGFDLKHKKDALALEKVKNKVDKSIKTRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 55 - protein, 629 aa, Staphylococcus aureus; identical to SEQ ID NO: 54

SEQ ID NO: 56 - protein, 629 aa, Staphylococcus aureus
MTFNEIIFKNFRQNLSHYAIYLFSLITSVVLYFSFVALKYAHKLNMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLITTGYYLSLNIVQYYDSIGTLMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKSKQPYNVTITSDKYIPSTDLKRGQADLFVAEGSIKDLVKHKKHGKAVIGTKKHHVNIKLRKDINKIYFMTDVDLGGPTFVLNDKDYQEIRKYTKAKHIVSQFGFDLKHKKDALALEKAKNKVDKSIETRSEAISSISSLTGILLFVTSFLGITFLIAVCCIIYIKQIDETEDELENYSILRKLGFTQKDMARGLKFKIMFNFGLPLVIALSHAYFTSLAYMKLMGTTNQIPVFIVMGLYICMYAVFAVTAYNHSKRTIRHSI

SEQ ID NO: 57 - protein, 608 aa, Artificial Sequence
  consensus from SACOL0720 orthologue alignment
  MISC_FEATURE (1)..(45): Xaa = any amino acid
  MISC_FEATURE (213)..(213): Xaa = any amino acid
  MISC_FEATURE (231)..(231): Xaa = any amino acid
  MISC_FEATURE (368)..(368): Xaa = any amino acid
  MISC_FEATURE (370)..(370): Xaa = any amino acid
  MISC_FEATURE (384)..(384): Xaa = any amino acid
  MISC_FEATURE (414)..(414): Xaa = any amino acid
  MISC_FEATURE (486)..(486): Xaa = any amino acid
  MISC_FEATURE (494)..(494): Xaa = any amino acid
  MISC_FEATURE (576)..(576): Xaa = any amino acid
XXXXXXXXXXXXXXXXXXXXXXXXXXXXXXXXXXXXXXXXXXXXXMTESYPIIKEGSQVGSYFLFFIIIAFLLYANVLFIKRRSYELALYQTLGLSKFNIIYILMLEQLLIFIITAILGIIIGIFGSKLLLMIVFTLLGIKEKVPIIFSLRAVFETLMLIGVAYFLTSAQNFILVFKQSISQMSKNNQVKETNHNKITFEEVVLGILGIVLIXTGYYLSLNIVQYYDSIGXLMFILLSTVIGAYLFFKSSVSLVFKMVKKFRKGVISVNDVMFSSSIMYRIKKNAFSLTVMAIISAITVSVLCFAAISRASLSSEIKYTAPHDVTIKDQQKANQLASELNNQKIPHFYNYKEVIHTKLYKDNLFDVKXKXPYNVTITSDKYIPXTDLKRGQADLFVAEGSIKDLVKHKKHGKAXIGTKKHHVNIKLRKDINKIYFMTDVDLGGPTFVLNDKDYQEIRKYTKAKHIVSQFGFDLKHKKDALALEKAXNKVDKSIXTRSEAISSISSLTGILLFVTSFLGITFLIAVCCIIYIKQIDETEDELENYSILRKLGFTQKDMARGLKFKIMFNFGLPLVIXLSHAYFTSLAYMKLMGTTNQIPVFIVMGLYIC

SEQ ID NO: 58 - protein, 243 aa, Staphylococcus aureus
MILSYLKIEFKVIMRKKTTLILSILFPVIFYILFTSILELPEDVKPKFYKEYMYSMTVYSLLSFSLLTFPLDIINEKQNEWRQRLMVTPFTFTSYYISKVVKTMLQFAIAILVIFMVGHFYKGVAMSAVQWLESGIFLWLGASLLITFGILFSLLNDIQKTSALANIVTIGLAVLGGLWFPINTFPNWLQHVAHVLPSYHLRKLGVDIASNHHINLISFAIILLYALGSIIAVYCISHFKRAE

SEQ ID NO: 59 - protein, 276 aa, Staphylococcus aureus
MHKIFSKNNLIFFVFVAFIFVVIVLQFFVSSENATKVNLSQTFEPISWLHLLGTDDYGRDLFTRIIIGARSTLFVTVLTLIAIVVIGVTLGLFAGYKKGWIERLVLRFIDVGLSIPEFIIMIALASFFQPSLWNLVISITLIKWMNYTRLTRSIVNSEMNKPYIKMAQLFHVPTRTILIRHLTPKIIPAIIVLMVVDFGKIILYISSLSFIGLGAQPPTPEWGAMLQQGRDFISSHPIMLIAPASVIAITILIFNLTGDALRDRLLKQRGEYDESH

SEQ ID NO: 60 - protein, 136 aa, Staphylococcus aureus
MNTNDAIKILKENGLKYTDKRKDMLDIFVEEDKYINAKYIQQVMDENYPGISFDTIYRNLHLFKDLGIIENTELDGEMKFRIACTNHHHHHFICEKCGDTKVIDYCPIDQIKLSLPGVNIHKHKLEVYGVCESCQD

SEQ ID NO: 61 - protein, 199 aa, Staphylococcus aureus
MAMNFKVFDNSQLVAEYAADIIRKQFNNNPTTIAGFHLDTDQAPVLDELKKNVEKHAVDFSQINILDYDDKKSYFEALGVPAGQVYPIAYEKDAIELIADKIKTKENKGKLTLQVVSIDEQGKLNVSIRQGLMEAREIFLVVTGANKRDVVEKLYQENGKTSFEPADLKAHRMVNVILDKEAAAGLPEDVKAYFTSRFA

SEQ ID NO: 62 - protein, 233 aa, Staphylococcus aureus
MTHKYISTQMLIIFTALMIIANFYYIFFEKIGFLLVLLLGCVLVYVGYLYFHKIRGLLAFWIGALLIAFTLLSNKYTIIILFVFLLLLIVRYLIHKFKPKKVVATDEVMTSPSFIKQKWFGEQRTPVYVYKWEDVQIQHGIGDLHIDLTKAANIKENNTIVVRHILGKVQVILPVNYNINLHVAAFYGSTYVNEKSYKVENNNIHIEEMMKPDNYTVNIYVSTFIGDVEVIYR

SEQ ID NO: 63 - protein, 260 aa, Staphylococcus aureus
MLIQLDQIGRMKQGKTILKKISWQIAKGDKWILYGLNGAGKTTLLNILNAYEPATSGTVNLFGKMPGKVGYSAETVRQHIGFVSHSLLEKFQEGERVIDVVISGAFKSIGVYQDIDDEIRNEAHQLLKLVGMSAKAQQYIGYLSTGEKQRVMIARALMGQPQVLILDEPAAGLDFIARESLLSILDSLSDSYPTLAMIYVTHFIEEITANFSKILLLKDGQSIQQGAVEDILTSENMSRFFQKNVAVQRWNNRFSMAMLE

SEQ ID NO: 64 - protein, 209 aa, Staphylococcus aureus
MKRLVTGLLALSLFLAACGQDSDQQKDGNKEKDDKAKTEQQDKKTNDSSKDKKDNKDDSKDVNKDNKDNSANDNQQQSNSNATNNDQNQTNNNQSSNNQANNNQKSSYVAPYYGQNAAPVARQIYPFNGNKNQALQQLPNFQTALNAANNEANKFGSNNKVYNDYSIEEHNGNYKYVFSFKDPNANGKYSIVTVDYTGQAMVTDPNYQQ

SEQ ID NO: 65 - protein, 93 aa, Staphylococcus aureus
MILNFNQFENQNFFNGNPSDTFKDLGKQVFNYFSTPSFVTNIYETDELYYLEAELAGVNKEDISIDFNNNTLTIQATRSAKYKSEQLILDERN

SEQ ID NO: 66 - protein, 439 aa, Staphylococcus aureus
MRTGHYTPIPNEPHYLVISHADKLTATEKAKLRLLIIKQKLDISLAESVVSSPIASEHVIEQLTLFQHERRHLRPKISATFLAWLLIFLMFALPIGIAYQFSDWFQNQYVSAWIEYLTQTTLLNHDILQHILFGDYGVLSLGTYSLVWALPVVILISLSTAIIDQTGLKSWMIWAIEPSMLWIGLQGNDIVPLLEGFGCNAAAISQAAHQCHTCTKTQCMSLISFGSSCSYQIGATLSIFSVAGKSWLFMPYLILVLLGGILHNRIWLKKNDQQLSVPLPYDRQLHMPNIRQMLLQMWQNIQMFIVQALPIFITICLIVSILSLTPILNVLSQIFTPILSLLGISSELSPGILFSMIRKDGMLLFNLHQGALLQGMTATQLLLLVFFSSTFTACSVTMTMLLKHLGGQSALKLIGKQMVTSLSLVIGVGIIVKIVMLII

SEQ ID NO: 67 - protein, 275 aa, Artificial Sequence
  SACOL0029-SACOL1867mut fusion
MHHHHHHLESREQLSVEEYETFFNRFDNQEFDFERELTQDPYSKVYLYSIEDHIRTYKIEKGGGGSGGGGSGGGGSTQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNAGSPVLNSNNEVIGVVYGGIGKIGSEYNGAVYFTPQIKDFIQKHIEQ

SEQ ID NO: 68 - skipped

SEQ ID NO: 69 - protein, 15 aa, Artificial Sequence
  Linker
GGGGSGGGGSGGGGS

SEQ ID NO: 70 - protein, 5 aa, Artificial Sequence
  Linker
ERKYK

SEQ ID NO: 71 - protein, 10 aa, Artificial Sequence
  Linker
EAAAKEAAAK

SEQ ID NO: 72 - protein, 5 aa, Artificial Sequence
  Linker
EAAAK

SEQ ID NO: 73 - protein, 15 aa, Artificial Sequence
  Linker
EAAAKEAAAKEAAAK

SEQ ID NO: 74 - protein, 10 aa, Artificial Sequence
  Linker
ERKYKERKYK

SEQ ID NO: 75 - protein, 15 aa, Artificial Sequence
  Linker
ERKYKERKYKERKYK

SEQ ID NO: 76 - protein, 5 aa, Artificial Sequence
  Linker
GGGGS

SEQ ID NO: 77 - protein, 10 aa, Artificial Sequence
  Linker
GGGGSGGGGS

SEQ ID NO: 78 - protein, 6 aa, Artificial Sequence
  Linker
  MISC_FEATURE (1)..(1): Xaa = any amino acid
  MISC_FEATURE (3)..(3): Xaa = any amino acid
  MISC_FEATURE (5)..(5): Xaa = any amino acid
XPXPXP

SEQ ID NO: 79 - protein, 12 aa, Artificial Sequence
  Linker
  MISC_FEATURE (1)..(1): Xaa = any amino acid
  MISC_FEATURE (3)..(3): Xaa = any amino acid
  MISC_FEATURE (5)..(5): Xaa = any amino acid
  MISC_FEATURE (7)..(7): Xaa = any amino acid
  MISC_FEATURE (9)..(9): Xaa = any amino acid
  MISC_FEATURE (11)..(11): Xaa = any amino acid
XPXPXPXPXPXP

SEQ ID NO: 80 - protein, 275 aa, Artificial Sequence
  SACOL-0029-SACOL1867 fusion
MHHHHHHLESREQLSVEEYETFFNRFDNQEFDFERELTQDPYSKVYLYSIEDHIRTYKIEKGGGGSGGGGSGGGGSTQVKDTNIFPYNGVVSFKDATGFVIGKNTIITNKHVSKDYKVGDRITAHPNGDKGNGGIYKIKSISDYPGDEDISVMNIEEQAVERGPKGFNFNENVQAFNFAKDAKVDDKIKVIGYPLPAQNSFKQFESTGTIKRIKDNILNFDAYIEPGNSGSPVLNSNNEVIGVVYGGIGKIGSEYNGAVYFTPQIKDFIQKHIEQ

SEQ ID NO: 81 - protein, 6 aa, Artificial Sequence
  poly-His tag
HHHHHH

SEQ ID NO: 82 - protein, 9 aa, Artificial Sequence
  RGS-His tag
RGSHHHHHH

SEQ ID NO: 83 - protein, 11 aa, Artificial Sequence
  RGS-His tag
RGSHHHHHHGS

SEQ ID NO: 84 - DNA, 720 bp, Staphylococcus aureus
atgaataaaaatatagtcattaaaagcatggcagcattagccattctaacctcagtaactggaataaatgctgcagtcgttgaagagacacaacaaatagcaaatgcagagaagaatgttacgcaagttaaagatacaaatatttttccatataatggcgtcgtttcatttaaagatgcgacaggttttgtaattggaaaaaatacaattatcaccaataaacatgtatcaaaagattataaagttggcgatagaattactgcccatccagacggtgacaaaggaaatggtggtatatataaaattaaaagcatttctgattatccgggtgatgaagacatctctgtcatgaatattgaagaacaagcagttgaacgtggaccaaaaggctttaattttaatgaaaatgtccaagcattaaattttgcgaaagatgctaaagttgatgacaaaattaaagttattggttacccattacctgctcaaaatagttttaaacagtttgaatctacaggaactataaaaagaatcaaagacaatattttaaattttgatgcatacattgaacccgggaattcaggatcaccagttctaaattctaacaatgaggtcataggtgtggtgtatggcggtattggaaaaattggttctgaatataatggtgccgtatactttacgcctcaaatcaaagattttattcaaaagcacattgaacaataa

SEQ ID NO: 85 - DNA, 720 bp, Staphylococcus aureus
atgaataaaaatatagtcattaaaagcatggcagcattagccattctaacctcagcaactggaataaatgctgcagtcgttgaagagacacaacaaatagcaaatgcagagaagaatgttacgcaagttaaagatacaaatatttttccatataatggcgtcgtttcatttaaagatgcgacaggttttgtaattggaaaaaatacaattatcaccaataaacatgtatcaaaagattataaagttggcgatagaattactgcccatccagacggtgacaaaggaaatggtggtatatataaaattaaaagcatttctgattatccgggtgatgaagacatctctgtcatgaatattgaagaacaagcagttgaacgtggaccaaaaggctttaattttaatgaaaatgtccaagcattaaattttgcgaaagatgctaaagttgatgacaaaattaaagttattggttacccattacctgctcaaaatagttttaaacagtttgaatctacaggaactataaaaagaatcaaagacaatattttaaattttgatgcatacattgaacccgggaattcaggatcaccagttctaaattctaacaatgaggtcataggtgtggtgtatggcggtattggaaaaattggttctgaatataatggtgccgtatactttacgcctcaaatcaaagattttattcaaaagcacattgaacaataa

SEQ ID NO: 86 - DNA, 720 bp, Staphylococcus aureus; identical to SEQ ID NO: 1

SEQ ID NO: 87 - DNA, 720 bp, Staphylococcus aureus; identical to SEQ ID NO: 1

SEQ ID NO: 88 - DNA, 720 bp, Staphylococcus aureus; identical to SEQ ID NO: 1

SEQ ID NO: 89 - DNA, 720 bp, Staphylococcus aureus
atgaataaaaatatagtcattaaaagcatggcagcattagccattctaacctcagtaactggaataaatgctgcagtcgttgaagagacacaacaaatagcaaatgcagagaagaatgttacgcaagttaaagatacaaataattttccatataatggcgtcgtttcatttaaggatgcgacaggttttgtaattggaaaaaatacaattatcaccaataaacatgtatcaaaagattataaagttggcgatagaattactgcccatccaaacggtgacaaaggaaatggtggtatatataaaattaaaagcatttctgattatccgggtgatgaagacatctctgtcatgaatattgaagaacaagcagttgaacgtggaccaaaaggctttaattttaatgaaaatgtccaagcattcaattttgcgaaagatgctaaagttgatgacaaaattaaagttattggttacccattacctgctcaaaatagttttaaacagtttgaatctacaggaactataaaaagaatcaaagacaatattttaaattttgatgcatacattgaacccgggaattcaggatcaccagttctaaattctaacaatgaggtcataggtgtggtgtatggcggtattggaaaaattggttctgaatataatggtgccgtatactttacgcctcaaatcaaagattttattcaaaagcacattgaacaataa

SEQ ID NO: 90 - DNA, 720 bp, Staphylococcus aureus; identical to SEQ ID NO: 89

SEQ ID NO: 91 - DNA, 720 bp, Staphylococcus aureus; identical to SEQ ID NO: 89

SEQ ID NO: 92 - DNA, 720 bp, Staphylococcus aureus; identical to SEQ ID NO: 89

SEQ ID NO: 93 - DNA, 720 bp, Staphylococcus aureus; identical to SEQ ID NO: 89

SEQ ID NO: 94 - DNA, 720 bp, Staphylococcus aureus
atgaataaaaatatagtcattaaaagcatggcagcattagccattctaacctcagtaactggaataaatgctgcagtcgttgatgagacacaacaaatagcaaatgcagagaagaatgttacgcaagttaaagatacaaatgtttttccatataatggcgtcgtttcatttaaagatgcgacaggttttgcaattgaaaaaaatacaattatcaccaataaacatgtatcaaaggactataaagttggcgatagaattactgcccacccaaatggtgacaaaggcaacggtggaatttataaaattaaaaatatttctgattatccaggtaatgaggatatatcagtaatgaacgttgaagaaaatgctgttgaacgtggagcaaatggttataattttaatgaaaatgtccaagcattcaattttgcgaaagatgctaaagttgacgacaaaattaaagttattggataccctttaccagctcaaaatacattcaaacaatttgaatcaactggaactgtaaaaagtattaaagataataatttaaattttgatgcatatatcgagcctggaaattcaggatccccagttttaaatttaaataatgaagtcgtaggagttgtgtatggaggtattggaaaaatcggatcagaatacaatggtgcagtttattttacgcctcaaatcaaagaatttattcaaaagcacattgaacaataa